(19) United States
(12) Patent Application Publication
(10) Pub. No.: US 2015/0329895 A1
(43) Pub. Date: Nov. 19, 2015

(54) Methods of Determining Cell Mediated Response

(71) Applicant: Advanced Medical Research Institute of Canada, Sudbury (CA)

(72) Inventors: Janet McElhaney, Sudbury (CA); Xin Zhou, Unionville, CT (US); Ernst Christiaan Soethout, MS Okijk (NL); Graham Pawelec, Tubingen (DE)

(21) Appl. No.: 14/375,854

(22) PCT Filed: Jan. 30, 2013
(86) PCT No.: PCT/CA13/00076

§ 371 Date: Jul. 31, 2014

Related U.S. Application Data
(60) Provisional application No. 61/592,833, filed on Jan. 31, 2012.

(51) Int. Cl. C12Q 1/37; C12N 7/00; A61K 39/145

(52) U.S. Cl. CPC C12Q 1/37; A61K 39/145; C12N 7/00; G01N 2333/96436; C12N 2760/16034; A61K 2039/525

(57) ABSTRACT

The invention relates to the use of one of more biomarkers, such as granzyme B, to determine an immune response, such as a cell-mediated immune response, of a subject to an immunostimulatory composition. The invention also relates to methods of treating a subject determined to develop a poor immune response to an immunostimulatory composition. Further, the invention relates to kits for use in practicing methods of the invention.

### CROSS REFERENCE TO RELATED APPLICATIONS

This application claims the benefit of and priority from U.S. Provisional Patent Application No. 61/592,833, filed on Jan. 31, 2012, which is incorporated herein by reference in its entirety.

### GOVERNMENT SUPPORT

The work was primarily sponsored by the Canadian Institutes for Health Research (CIHR). This work was also partly sponsored by the National Institutes of Health (NIH), National Institute of Allergy and Infectious Diseases, R01 AI68265. The study was partly conducted through the Lowell P. Weicker, Jr. General Clinical Research Center funded by the NIH, National Center for Research Resources (Grant Number MO1 RR06192) at the University of Connecticut Health Center (UCHC), and in collaboration with the UConn Center on Aging. Accordingly, the United States government may have certain rights in this invention.

### FIELD OF THE INVENTION

The present invention relates to methods of predicting or determining subject's ability to develop a cell-mediated immune response, methods to select the subject for treatment, methods to treat the subject, and kits for practicing methods of the invention.

### BACKGROUND

The need for more effective vaccines and the need for methods to predict vaccine efficacy are well recognized, but there have been significant challenges. For example, use of antibody responses as a sole predictor of vaccine efficacy is limited; and changes of the immune system that occur with age may affect the effectiveness of a vaccine.

For example, in the case of influenza, it is recognized that serum antibody titers against different influenza strains do not distinguish those older individuals who subsequently develop influenza illness from those who do not [7, 8, 25]. Prior exposure at a younger age (and thus the presence of immune memory) has been suggested as a mechanism for lower attack rates for both pandemic and seasonal H1N1 influenza compared to A/H3N2 strains in older adults. However, the serious complication rates of influenza infection in older adults are similar across the different subtypes of influenza A and thus at a population level, influenza A/H3N2 has had a greater impact in older adults relative to seasonal H1N1 [3] and pandemic H1N1 [26].

A multitude of changes in the immune system occur with aging. For example, the specific mechanisms that increase risk for influenza illness and limit the protective effects of vaccination in older adults are poorly understood. The importance of T-cell mediated clinical protection against influenza in older adults is increasingly recognized and has underscored the importance of including cellular immune measures in the assessment of vaccine efficacy in the over 65 population[25, 26]. Age-related changes in T cell responses may be associated with a decline in the antibody response to influenza vaccination[27, 28] but mechanistic links have not been made nor have these changes been correlated with protection against influenza. These observations show that the traditional role of vaccines in providing antibody-mediated protection against infection or “sterilizing immunity” in young adults, is replaced by T-cell mediated clearance of the virus once infection occurs, thus providing “clinical protection” against disease in older adults.

Accordingly, there remains a need for methods to determine a subject's ability to develop an effective immune response.

### SUMMARY OF THE INVENTION

The invention relates to methods of determining a subject's ability to develop an immune response, such as a cell-mediated immune response.

In one aspect, the invention relates to a method of predicting an immune response of a subject to an immunostimulatory composition, the method comprising (a) determining a level of one or more biomarkers in a test sample from the subject, and (b) comparing the level of the one or more biomarkers in the test sample to a level of one or more biomarkers in a control sample, wherein a change in the level of the one or more biomarkers in the test sample relative to the control sample is indicative of the subject's immune response to the immunostimulatory composition.

In another aspect, the invention relates to a method of determining an immune response of a subject to an immunostimulatory composition, the method comprising (a) immunizing the subject with the immunostimulatory composition, (b) determining a proportion of cells that express one or more biomarkers in a test sample that was obtained from the subject and stimulated with the immunostimulatory composition, (c) comparing the proportion of cells that express the one or more biomarkers in the test sample to a proportion of cells that express one or more biomarkers in a control sample, and (d) determining a strength of the immune response to the immunostimulatory composition by detecting a difference in the proportion of cells that express the one or more biomarkers in the test sample and the control sample.

In another aspect, the invention relates to a method for determining an immune response of a subject to an immunostimulatory composition, the method comprising: (a) determining a first level of granzyme B (bGrzB) activity from a first sample from the subject, (b) administering the immunostimulatory composition to the subject, (c) determining a second level of granzyme B (tGrzB) activity from a second sample from the subject, and (d) calculating an induced level of granzyme B (iGrzB) activity, wherein iGrzB activity=fold increase in tGrzB activity over bGrzB activity, wherein, a high level of bGrzB activity, a low level of iGrzB activity or a low level of tGrzB activity indicates that the subject is at risk of developing a poor immune response to the immunostimulatory composition, and wherein a low level of bGrzB activity or a high level of iGrzB activity indicates that the subject is at risk of developing a strong immune response to the immunostimulatory composition.

In another aspect, the invention relates to a method of determining an immune response of a subject to an immunostimulatory composition, the method comprising: (a) immunizing the subject with the immunostimulatory composition, (b) determining a first level of granzyme B (bGrzB) activity in a test sample from the subject, (b) stimulating the test sample with the immunostimulatory composition, (c) determining a second level of granzyme B (tGrzB) activity from the test sample stimulated with the immunostimulatory composition, and (d) calculating an induced level of granzyme B (iGrzB) activity, wherein iGrzB activity is equal to fold increase in tGrzB activity over bGrzB activity, (e) comparing the iGrzB activity in the test sample to an iGrzB activity of a control sample, wherein a decrease in the level of bGrzB activity and/or an increase in the level of iGrzB activity in the test sample compared to the control sample indicates development of a strong immune response in the subject to the immunostimulatory composition, and wherein a similar or an increase in the level of bGrzB activity and/or a similar or a decrease in the level iGrzB activity in the test sample compared to the control sample indicates development of a poor immune response in the subject to the immunostimulatory composition.

In another aspect, the invention relates to a method of treating a subject in need thereof, the method comprising (a) identifying the subject determined to develop a poor immune response to an immunostimulatory composition according to a method of the invention, and (b) treating the subject with the immunostimulatory composition according to an altered immunization schedule; and/or treating the subject with an enhanced immunostimulatory composition, and/or treating the subject with a therapeutic agent.

In another aspect, the invention relates to a method of treating a subject in need thereof, the method comprising (a) determining a level of granzyme B activity in a test sample from the subject, and (b) if the level of granzyme B activity in the test sample is greater than a level of granzyme B activity in a chronic disease negative population, then (i) treating the subject with an enhanced immunostimulatory composition, and/or (ii) treating the subject with the immunostimulatory composition according to an altered immunization schedule, and/or treating the subject with a therapeutic agent.

In another aspect, the invention relates to a kit for determining the response of a subject to an immunostimulatory composition, the kit comprising a substrate for granzyme B, and/or one or more binding agents selective for CMV, granzyme B, perforin, CD45RA, CD4, CD8, CCR7, CD25 and/or CD127.

Other aspects and features of the present invention will become apparent to those of ordinary skill in the art upon review of the following description of specific non-limiting embodiments of the invention in conjunction with the accompanying tables and figures.

### BRIEF DESCRIPTION OF THE DRAWINGS

FIG. 1. Shows a phenotype shift and expression of granzyme B (GzmB) and perforin (Perf) delineates age-related changes in different T cell subsets from PBMCs from healthy young (HY) and older (HO, 60-70 years old, and HO, >80 years old (yo)) adults (n=15/group) obtained at 4 weeks or 10 weeks post vaccination. (A) CD4+ and CD8+ subsets were defined by the expression of cell surface molecules, CD45RA and CCR7, and their intracellular cytolytic effector molecules, GzmB and Perf. The percentage of cells within each of the T-cell subsets: CD45RA+CCR7+ (Naïve), CD45RA-CCR7+(central memory, CM), CD45RA−CCR7− (effector memory, EM), and CD45RA+CCR7− (Effector) are shown. The proportion of naïve, memory and effector T cell subsets that are (B) CD4+ or (C) CD8+ are shown. The proportion of GzmB+Perf+ effector T cells in (D) CD4+, and (E) CD8+ subsets at 4 weeks post-vaccination and at (F) 10 weeks post vaccination in both CD4+ and CD8+ T cell subsets are also shown. Error bars represent standard error of the mean. Significant differences in the means between age groups are shown as *p<0.0001, **p=0.001, and ***p<0.001.

FIG. 2 shows degranulating activity in GzmB+ T-cells responding to influenza virus in young (HY) and older (HO, >65 years old) adults. Graphs show the proportion of cells expressing the degranulating marker CD107a in the CD4+ and CD8+ T cells from young and older adults. Error bars represent standard error of the mean. Significant differences in the means between age groups are shown as ***p<0.001.

FIG. 3 shows a distribution of effector CD4+ T cells and CD8+ T cells in older adults (HO, >65 years old) responding to influenza virus, demonstrated by cytolytic activity, compared to young adults (HY) at (A) 4-weeks and (B) 10-weeks post-vaccination. Cytotoxicity was calculated as mean % specific lysis for Effector:Target ratios of 10:1. Results are shown for each of the CD4+ and CD8+ subsets, and differences between age groups are shown as *p<0.01, **p<0.005. Error bars represent standard error of the mean.

FIG. 4 shows a comparison of the cytotoxic effector T-cell response to seasonal A/H3N2 and pandemic influenza H1N1 (pH1N1) virus in young (HY) and older adults (HO, 60-70 yo or >80 yo) who received seasonal influenza split-virus vaccine. (A) In HY, CD4+ and CD8+ subsets that were GzmB+Perf+ were defined by the expression of cell surface molecules, CD45RA, CCR7, CD25, CD127. Differences in the response to pH1N1 vs. A/H3N2 are shown as ***p<0.001, *p<0.01, and **p<0.005 in (B) through (F). The effector (CD45RA+CCR7+CD127+) subsets of CD4+ and CD8+ for (B) CD25+ and (C) CD25− T cells are shown for HY. (D) Proliferation assay of CD4+ or CD8+CD45RA+CCR7−CD25+CD127+ and CD45RA+CCR7−CD25−CD127+ T-cell subsets sorted from PBMCs from HY stimulated with influenza A/H3N2 or pH1N1 live virus for 5-6 days. (E) Cytotoxicity assay of T cell subsets sorted from PBMCs from young adults stimulated with live virus for 5 days. Cytotoxicity was calculated as mean % specific lysis for Effector:Target ratios of 5:1. (F) T cell responses of effector (CD45RA+CCR7+CD25+CD127+) cells sorted from PBMCs from HY and HO, 60-70 yo and >75 yo, stimulated for 5-6 days with A/H3N2 or pH1N1Error bars represent standard error of the mean.

FIG. 5 shows a distribution of human PBMC subsets from healthy young (HY) and older (HO, 60-70 years old, and HO, >80 years old) adults (n=15/group) obtained at the time before vaccination were cultured with live influenza virus for 20 hr. CD4+ and CD8+ subsets were defined by the expression of cell surface molecules, CD45RA and CCR7, and their intracellular cytolytic effector molecules, GzmB and Perf. The percentage of cells within effector T-cell subset CD45RA+CCR7− are shown. Error bars represent standard error of the mean.

FIG. 6 shows the results of a proliferation assay of CD4+ or CD8+, and their effector subsets (CD45RA+CDR&−) sorted from human PBMCs of healthy young (HY) and older (HO, 75-85 years old) adults (n=6/group) stimulated with influenza seasonal A/H3N2 live virus for 5-6 days

FIG. 7 shows baseline granzyme B (bGrzB) activity in unstimulated PBMC lysates from older adults, including those who were healthy and those with diabetes, who were CMV+ or CMV−. CMV status was determined by serology; CMV+(n=21) vs. CMV− (n=7). Error bars represent standard error of the mean. Difference between the CMV+ and CMV-subsets is p=0.029. The presence of diabetes was not significant in the analysis (data not shown).

FIG. 8. (A) GrzB activity in unstimulated CD3+ T cells (bGrzB) in lysates from older adults, including those who were healthy and those with diabetes, who were CMV+ or CMV−. Error bars represent standard error of the mean. Difference between the CMV+ and CMV− subsets is p=0.001. (B) induced GrzB activity [(fold increase in tGrzB activity over bGrzB activity)] in response to influenza challenge pre-vaccination, and at 4, 10 and 20 weeks post vaccination. Difference between the CMV+ and CMV− subsets is p=0.01. Results presented in base 10 log transformation.

### DETAILED DESCRIPTION

The present invention relates to biomarkers, and uses thereof, for determining a subject's ability to develop an immune response. In various aspects, the invention relates to methods to determine an immune response of a subject to an immunostimulatory composition, methods of treating a subject in need thereof, and to kits for practicing the methods described herein.

In one aspect, the invention relates to a method of predicting an immune response of a subject to an immunostimulatory composition, where the method comprises (a) determining a level of one or more biomarkers in a test sample from the subject, and (b) comparing the level of the one or more biomarkers in the test sample to a level of one or more biomarkers in a control sample. A change in the level of the one or more biomarkers in the test sample relative to the control sample is indicative of the subject's immune response to the immunostimulatory composition.

In one embodiment, the methods described herein comprise determining a level of one or more biomarkers in test sample. This may be achieved using conventional assays and techniques, and may depend on the biomarker, as later on described.

As used herein, a “biomarker” refers to a biomolecule, such as a nucleic acid, protein or protein fragment present in a test sample from a subject, where the quantity, concentration or activity of the biomarker in the sample is useful in providing information about the ability of the subject to develop an immune response. A biomarker may also refer to a physiological condition in an individual that is derived from the biomolecule.

As used herein, a “level of one or more biomarkers” refers to the protein, nucleic acid, or activity levels of the one or more biomarkers. In other embodiments, the level of one or more biomarkers refers to the absolute amount, concentration, or level of the biological activity of the one or more biomarkers. In another embodiment, the level of one or more biomarkers is indicative of severity of a condition measured by the one or more biomarkers. Optionally, the phrase “level of one or more biomarkers in a control sample” refers to a predetermined value or threshold of a biomarker or levels or more than one biomarker, such as a level or levels known to be useful for distinguishing between subjects who will develop a strong immune response to an immunostimulatory composition from those who will not. Optionally, the phrase “level of one or more biomarkers in a control sample” refers to determining a level of a biomarker in a control sample run against the test sample.

In one embodiment, the methods described herein comprise comparing the level of one or more biomarkers in a test sample to a level of one of more biomarkers in a control sample. Optionally, the level of the biomarkers in the control sample is a predetermined or standardized level or threshold. For example, in one embodiment the level of the one or more biomarkers in the test sample is compared to one or more previously determined control levels. The process of comparing levels of biomarkers may also include determining the fold difference (either fold increase or fold decrease) in the level of the biomarkers in the test sample compared to the control sample.

As used herein, the term “control sample” refers to a sample representative of one or more subjects whose status with respect to immune response and/or biomarker levels are known. A skilled person would appreciate that the specific control sample may be chosen on the basis of the biomarker used. In one embodiment, the control sample is representative of healthy, typically older adult, subjects who do not suffer from a chronic disease (i.e. chronic disease negative), as defined herein. In another embodiment, the control sample is representative of subjects who are healthy young adults. In a further embodiment, the control sample represents subjects who are positive for a chronic disease. Optionally, the control sample is age-matched or matched for ethnicity or genetic background with the subject who provides the test sample.

An increase or decrease in the levels, or fold difference, of the biomarkers compared to the control level is predictive of the immune response in the subject to the immunostimulatory composition. In one embodiment the level of the one or more biomarkers in the test sample is compared to a threshold control level wherein an increased or decreased level in the test sample is predictive of the immune response in the subject to the immunostimulatory composition. In another embodiment, the magnitude of the difference between the level of the one or more biomarkers in the test sample from a subject and the one or more control levels is predictive of the strength of the immune response in the subject to the immunostimulatory composition.

In one embodiment, a difference in the level of the one or more biomarkers in the test sample compared to the control samples is used to predict the strength of the immune response that the subject will develop to the immunostimulatory composition. For example, in one embodiment an increase in the level of one or more biomarkers selected from granzyme B, cytomegalovirus (CMV), Epstein Barr virus (EBV), human immunodeficiency virus (HIV) or Herpes zoster in the test sample compared to a control sample predicts that the subject will develop a poor immune response to the immunostimulatory composition. In another embodiment, a decrease in the level of one or more biomarkers selected from granzyme B, CMV, EBV, HIV or Herpes zoster in the test sample compared to a control sample predicts that the subject will develop a strong immune response to the immunostimulatory composition. In one embodiment, the one or more biomarkers are granzyme B, optionally granzyme B activity, and CMV. In another embodiment, other biomarkers that may be of use in the methods described herein include biomolecules for assessing frailty.

In an embodiment of the invention, the level of one or more biomarkers that predicts that the subject will develop a poor immune response to the immunostimulatory composition, is at least: 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.2, 4.4, 4.6, 4.8, 5.0, 5.5, 6.0, 6.5, 7.0, 7.5, 8.0, 8.5, 9.0, 9.5, 10, 12, or 15 fold increased in the test sample compared to the control sample, wherein the control sample is healthy, typically older, adults that are chronic disease negative with respect to granzyme B; and wherein the control sample is healthy older adults with respect to CMV, EBV, HIV or Herpes zoster.

In an embodiment of the invention, the level of one or more biomarkers that predicts that the subject will develop a strong immune response to the immunostimulatory composition, is similar between the test sample and the control sample, or at least about: 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.2, 4.4, 4.6, 4.8, 5.0, 5.5, 6.0, 6.5, 7.0, 7.5, 8.0, 8.5, 9.0, 9.5, 10, 12, or about 15 fold decreased in the test sample compared to the control sample, wherein the control sample is healthy, typically older, adults that are chronic disease negative with respect to granzyme B; and wherein the control sample is healthy older adults with respect to CMV, EBV, HIV or Herpes zoster. As used herein, a similar level of one or more biomarker in the test sample compared to the control sample refers to a difference of no greater than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23% 24% or about 25%.

In embodiments of the invention, the biomarker is granzyme B. Granzyme B (also known as GrB, GzmB, GrmB, GrzB, GZMB, granzyme 2, cytotoxic T-lymphocyte-associated serine esterase 1) is a serine protease that in humans is encoded by the GZMB gene. Granzyme B proteins, peptides, fragments thereof and variants thereof are known in the art (see, for example, EAW66002.1 GI: 119586406; EAW66003.1 GI: 119586407). Granzyme B is expressed by cytotoxic T lymphocytes and natural killer cells.

Granzyme B may play a role in chronic infections. For example, terminally differentiated T cells have been observed in chronic cytomegalovirus (CMV) infection, and accumulate with age. Granzyme B activity in T cells has been observed to be increased in older adults who are seropositive, compared to those who are seronegative for CMV infection.

As described herein, levels of granzyme B varies with age. For example, older adults, relative to younger adults, have higher levels of granzyme in lysates of unstimulated peripheral blood mononuclear cells (PBMC). Flow cytometry studies have further demonstrated that a high proportion of GrzB-positive T cells are found in older adults compared to younger adults, and that these cells were non-cytolytic in the absence of perforin (Perf). These GrzB-positive T cells degranulated in the absence of stimulation and may represent terminally differentiated T cells

Without wishing to be bound by theory, CMV-specific T cells that accumulate with aging in response to chronic CMV infection and are associated with inflammation, contain active GrzB and are continuously degranulating and releasing active GrB into the circulation and the tissues of the subject. This baseline level of GrB activity (bGzmB, bGrB) may be useful as a measure of the accumulated effect of chronic viral or bacterial infection.

Again, without wishing to be bound by theory, bGzmB activity may have a negative effect on the response to vaccination (e.g. influenza vaccination) in older adults. Total GrB (tGrB, tGzmB, tGrmB) activity measured in virus stimulated PBMC is the combined activity of bGrB and the induced GrB activity (iGrB, iGzmB, iGrzB) derived from cells that become GrB positive and perforin positive in response to virus stimulation—where iGrB activity is the fold increase in tGrB activity over bGrB activity.

In an embodiment of the invention, baseline granzyme B activity refers to a level of GrB activity (U/mg protein) in a subject, or in a sample from a subject, before administration of an immunostimulatory composition. Baseline granzyme B activity may also refer to a level of GrB activity in a subject, or sample from a subject, where the subject has not been administered an immunostimulatory composition for at least 3 months, at least 4 months, at least 5 months, at least 6 months, at least 7 months, at least 8 months, at least 9 months, at least 10 months, at least 11 months, at least 12 months, at least 18 months, at least 24 months, at least 36 months, at least 48 months, or at least 60 months prior to the date the level of granzyme B activity in the subject was assessed. In an embodiment of the invention, baseline granzyme B activity refers to a level of granzyme B activity in a subject, or in a sample from a subject, where the subject has a chronic disease, and the subject may or may not be receiving, or have received, treatment for the disease. In another embodiment, baseline granzyme B activity is the activity (U/mg protein) detected in lysates of unstimulated T cells purified from the subject, or a sample of the subject.

In one embodiment, total GrB activity (tGrB) refers to the level of GrB activity in a subject, or in a sample from a subject, following administration of an immunostimulatory composition, and sufficient time for the subject's immune system to mount a response to the immunostimulatory composition. This time may be from about 1 day to about 6 months, or anytime therebetween. More particularly, the time may be 2, 3, 4, 5, 6, 7, 8, 9, 10, 11, 12, 13, 14, 15, 16, 17, 18, 19, 20, 21, 22 or 23 weeks, or any time therebetween. Total granzyme B activity is optionally detected as the granzyme B activity in lysates of stimulated T cells (for example, by stimulation with an immunostimulatory composition, a pathogen (e.g. a viral pathogen), or some other substance).

In one embodiment, induced GrB activity (iGrB) is the amount of GrB activity resulting from the subject's immune response to the immunostimulatory composition, and may be obtained by subtracting bGrB from tGrB. In another embodiment, induced granzyme B activity is the total granzyme B activity (i.e. the granzyme B activity detected in lysates of stimulated T cells (e.g. by an immunostimulatory composition) minus the baseline granzyme B activity (i.e. granzyme B activity detected in lysates of unstimulated T cells).

In one embodiment, the level of granzyme B activity in a test sample is compared to the level of granzyme B activity in a control sample. In certain embodiments, the control sample may represent subjects who are without a chronic disease and/or who is an older adult. In other embodiments, the control sample may represent subjects who have a chronic disease and/or who is a younger adult.

As used herein, a “chronic disease” refers to, without limitation, a health condition, infection or disease that is persistent or otherwise long-lasting in its effects, for example lasting at least: 1, 5 or 10 years. For example, a chronic disease may include a chronic bacterial infection or a chronic viral infection; such as, a chronic infection by cytomegalovirus (CMV), Epstein Barr virus, Herpes zoster or HIV. A chronic disease may also include, without limitation, arthritis, asthma, cancer, COPD, AIDS and congestive heart failure. As used herein, a chronic disease negative population is a group of two or more subject, each of whom is negative for a chronic disease. As described herein, a cytomegalovirus (CMV) negative subject is a subject that is seronegative for CMV, when assessed by conventional methods. A cytomegalovirus (CMV) positive subject is a subject that is seropositive for CMV, when assessed by conventional methods. For example, CMV antibodies in a sample may be detected and/or quantified or by a kit, for example the ImmunoComb II from Orgenics, Is-CMV IgG Test Kit from DIAMEDIX, or the ToRCH.Serology kit from BioRad.

In one embodiment, the biomarker is granzyme B, and an increase in the baseline granzyme B activity in the test sample compared to the control sample is predictive of development of a poor immune response in the subject to an immunostimulatory composition.

In one embodiment, a chronic disease negative population may have a level of baseline granzyme B activity that is about 300 U/mg, about 290 U/mg, about 280 U/mg, about 270 U/mg, about 260 U/mg, about 250 U/mg, about 240 U/mg, about 230 U/mg, about 220 U/mg, about 210 U/mg, about 200 U/mg, about 190 U/mg, about 180 U/mg, or less of granzyme B.

In another embodiment, a level of baseline granzyme B activity greater than about 300 U/mg, greater than about 325 U/mg, greater than about 350 U/mg, greater than about 375 U/mg, greater than about 400 U/mg, greater than about 425 U/mg, greater than about 450 U/mg, greater than about 475 U/mg, greater than about 500 U/mg, greater than about 525 U/mg, greater than about 550 U/mg, greater than about 575 U/mg or greater than about 600 U/mg may be indicative of the subject's inability, or poor ability, to develop a cell-mediated response.

In embodiments of the invention, the method of determining a subject's ability to develop a cell-mediated immune response comprises determining a level of granzyme B activity in a sample from a subject, wherein the level of baseline granzyme B activity that is greater than a level of granzyme B of a CMV negative population at a 95% confidence interval, or that is greater than 300 U/mg, is indicative of the subject's inability, or poor ability, to develop a cell-mediated immune response.

In another embodiment, an increase in the level of the baseline granzyme B activity in the test sample compared to a sample from a chronic disease negative population, by at least 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 fold predicts development in the subject of poor immune response to the immunostimulatory composition.

In certain embodiments of the invention, the biomarker is CMV, EBV, HIV or Herpes zoster; typically CMV. In one embodiment, an increase in the level of CMV in a test sample of the subject, compared to a healthy older adult control, predicts development in the subject of a poor immune response to an immunostimulatory composition. In another embodiment, a similar level or a decrease in the level of CMV in a test sample compared to a control sample from a healthy older adult predicts development in the subject of a strong immune response to an immunostimulatory composition.

Comparison of the level of one or more biomarkers in a test sample to a level of one of more biomarkers in a control sample may be performed by methods known in the art. For example, in one embodiment the levels of individual biomarkers, such as granzyme B and CMV, are compared to predict the strength of a subject's immune response to an immunostimulatory composition. In another embodiment, levels from more than one biomarker are compared to predict the strength of a subject's immune response to an immunostimulatory composition.

A person skilled in the art will appreciate that a number of different methods may be useful to determine the level of the one or more biomarkers described herein. In one embodiment, the level of the biomarkers of the invention may be determined by nucleic acid amplification (e.g. real time PCR or other methods known in the art for determining RNA levels from gene expression). In one embodiment, the level of the biomarkers may be determined based on enzymatic reactions, (e.g. a substrate of a biomarker). For example, a substrate such as IEPDpna may be used to determine the activity of granzyme B. In another embodiment, the level of the biomarkers may be determined on the basis of protein expression, for example, through the use flow cytometry to identify protein in cells or a body fluid, such as blood. In one embodiment, protocols for determining the level of biomarkers use agents that bind to the biomarker protein of interest (i.e. binding agents). In one embodiment the binding agents are apatamers. In another embodiment, the binding agents are antibodies or antibody fragments. The term “aptamer” refers to oligonucleic acid or peptide molecules that bind to a specific target molecule. Typically, aptamers are single-stranded DNA or RNA (ssDNA or ssRNA) molecules that can bind to pre-selected targets including proteins and peptides with high affinity and specificity. Aptamers can assume a variety of shapes due to their propensity to form helices and single-stranded loops, and thus are useful in binding to diverse targets. The term “antibody” as used herein is intended to include monoclonal antibodies, polyclonal antibodies, chimeric antibodies, and single domain antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. The binding agents described herein may be labeled (e.g. radiolabeled, fluorescent labeled) for use in the methods of the invention.

A person skilled in the art will appreciate that a number of other methods may be useful to determine the levels one or more biomarkers in a sample, including immunoassays such as Western blots, ELISA, and/or immunoprecipitation followed by SDS-PAGE immunocytochemistry etc. Other embodiments include the use of methods for determining levels of a biomarker in a sample such as lateral flow and related immunochromatic tests used in point-of-care tests. In addition, nucleic acid or protein arrays (including microarrays) may be useful. For nucleic acid biomarkers such as mRNA, RT-PCR or quantitative RT-PCR or other methods known in the art for detecting and/or quantifying nucleic acids may also be useful.

In embodiments of the invention, a level of granzyme B may refer to the mRNA, protein, activity or any other measurable level of granzyme B in any form. Standard approaches, techniques and kits may be used to detect and/or measure granzyme B mNRA and protein levels, and granzyme B activity. For example, kits are available for analysis of GrB in plasma (e.g. ELISA kits from Bender Medsystems, or kits using other methods from conventional suppliers such as Research Diagnostics Inc, AbCam, RND Systems or the like). Such kits may comprise specific reagents for quantitative detection of GrB in the sample, and/or substrates for assaying the level of GrB enzyme activity in the sample. As an example, an IEPDpna substrate specific for GrzB (IEPD peptide with paranitroanilide chromophore) is combined with a plasma or serum sample, or a blood cell lysate. GrzB present in the sample cleaves the IEPDpna substrate and releases the pna, causing a colorimetric change that may be measured using a spectrophotometer.

In embodiments of the invention, the chronic disease is a chronic CMV infection; and a positive CMV serology may be indicative of the subject's inability, or poor ability, to develop a cell-mediated response.

As used herein, a “subject” refers to, without limitation, an animal, such as a mammal, and includes, without limitation, a human, primate, bird, water fowl, migratory bird, quail, duck, goose, poultry, chicken, swine, sheep, equine, horse, camel, canine, dog, feline, cat, tiger, leopard, civet, mink, stone marten, ferret, house pet, cow, livestock, rabbit, mouse, rat, guinea pig or other rodent, seal, whale and the like. The subject may be healthy, in that there are no symptoms of an infection, e.g. a bacterial infection or a viral infection, such as an influenza virus infection. However, a subject that exhibits no clinical symptoms of an infection, e.g. a bacterial infection or a viral infection, such as an influenza virus infection or a CMV infection, but is seropositive for the infectious agent, e.g. influenza virus or CMV, would not be considered a healthy subject. The subject may be immunologically naive with respect to a particular antigen or group of antigens, or the subject may have been previously exposed to a particular antigen or group of antigens. In embodiments of the invention, the subject may be infected with influenza virus.

A subject may be an adult human. An adult human subject may be a young adult (younger than 60 years of age, younger than 55 years of age, younger than 50 years of age, younger than 45 years of age, younger than 40 years of age, younger than 35 years of age, younger than 30 years of age, younger than 25 years of age, or younger than 20 years of age, typically about 20 to about 40 years of age, or any amount there between); or an older adult (60 or greater years of age, or any amount thereafter, for example, greater than 65 years of age, greater than 70 years of age, greater than 75 years of age, greater than 80 years of age, greater than 85 years of age, greater than 90 years of age, greater than 95 years of age, or greater than 100 year of age, typically about 60 to about 80 years of age, or about 60 to about 70 years of age, or about 80 years of age to about 100 years of age, or about 85 years of age to about 100 years of age, or about 80 years age or greater).

A subject may be a non-human adult animal. An adult non-human animal subject may be an older adult—that is, a non-human animal with age that is more than ¾ of the average lifespan of the non-human animal; or a younger adult—that is, a non-human animal with age that is between ¼ and ¾ of the average lifespan of the non-human animal.

As used herein, a “sample” refers generally to, without limitation, any biological substance from a subject that contains a biomolecule useful in the methods disclosed in this application. Non limiting examples of a sample include a body fluid, tissue or organ sample from a subject. For example, the sample may be a body fluid such as blood, serum, plasma, lymph fluid, urine or saliva. A tissue or organ sample, such as a non-liquid tissue sample may be digested, extracted or otherwise rendered to a liquid form—examples of such tissues or organs include, without limitation, cultured cells, blood cells, skin, liver, heart, kidney, pancreas, islets of Langerhans, bone marrow, blood, blood vessels, heart valve, lung, intestine, bowel, spleen, or the like. A plurality of samples may be collected at any one time. One or more samples may be taken from a subject at any time, for example before administration of a therapeutic agent or an immunostimulatory composition, at one or more points during a course of administration of a therapeutic agent or immunostimulatory composition, or at one or more points following a course of administration of a therapeutic agent or immunostimulatory composition

In embodiments of the invention, the methods described herein may be useful in determining an immune response of a subject to an immunostimulatory composition. As used herein, an “immune response” generally refers to a response of the immune system, including, for example, the adaptive immune system. An adaptive immune system generally comprises a humoral response and/or a cell-mediated response. The immune response may be a poor immune response or a strong immune response.

As used herein, a “poor” immune response refers to the subject developing either no detectable increase in immune response or an immune response that is reduced compared to a control subject, which will vary depending on the biomarker at issue, but includes, as applicable, a healthy young adult subject, a healthy older adult subject, or a subject that is without a chronic disease. In one embodiment, a poor immune response may be predicted having regard to a threshold level of granzyme B activity. In one embodiment, a poor immune response does not correlate with protection against the pathogen against which the immunostimulatory composition provided is directed.

As used herein, a “strong” immune response refers to the subject developing an immune response that is equal, or substantially equal, to a control subject, which will vary depending on the biomarker at issue, but includes, as applicable, a healthy young adult subject, a healthy older adult subject, or a subject that is without a chronic disease. In one embodiment, a strong immune response may be predicted having regard to a threshold level of granzyme B activity. In one embodiment, a strong immune response correlates with protection against the pathogen against which the immunostimulatory composition provided is directed.

An immune response to an immunostimulatory composition may be readily determined by conventional methods. For example, the immune response may be measured by assaying antigen specific lymphocytes, e.g. by assessing their proliferation, activity, or cytokine production. This may be achieved by many techniques, including without limitation, flow cytometry, ELISPOT, ELISA, intracellular staining, challenge studies.

A humoral response is the aspect of immunity that is mediated by secreted antibodies, produced in the cells of the B lymphocyte lineage (B cell). Secreted antibodies bind to antigens on the surfaces of invading microbes (such as viruses or bacteria), which flags them for destruction. Humoral immunity refers generally to antibody production and the processes that accompany it, as well as the effector functions of antibodies, including, for example, Th2 cell activation and cytokine production, memory cell generation, opsonin promotion of phagocytosis, pathogen elimination and the like.

A cell-mediated response generally refers to an immune response that does not involve antibodies but rather involves the activation of one or more of macrophages, natural killer cells (NK), antigen-specific cytotoxic T-lymphocytes (CD4+ or CD8+), and the release of various cytokines in response to an antigen. Cell-mediated immunity is used generally to refer to some Th cell activation, Tc cell activation and T-cell mediated responses. Cell mediated immunity is of particular importance in responding to intracellular pathogens, for example, viruses.

An immune response may be detected or measured by way of, for example, lymphocyte activity or levels, by method, approaches and techniques known in the art. For example, induction of antigen specific CD8 positive T lymphocytes may be measured using an ELISPOT assay; stimulation of CD4 positive T-lymphocytes may be measured using a proliferation assay. Antibody titers may be quantified using an ELISA assay; isotypes of antigen-specific or cross reactive antibodies may also be measured using anti-isotype antibodies (e.g. anti-IgG, IgA, IgE or IgM).

A hemagglutination inhibition (HI, or HAI) assay may be used to demonstrate the efficacy of antibodies induced by a vaccine or vaccine composition to inhibit the agglutination of red blood cells (RBC) by recombinant HA. Hemagglutination inhibitory antibody titers of serum samples may be evaluated by microtiter HAI (Aymard et al 1973). Erythrocytes from any of several species may be used—e.g. horse, turkey, chicken or the like.

Cross-reactivity HAI titres may be used to demonstrate the efficacy of an immune response to other strains of virus related to the vaccine subtype. For example, serum from a subject immunized with a vaccine composition of a first strain may be used in an HAI assay with a second strain of whole virus or virus particles, and the HAI titer determined.

Cytokine presence or levels may be quantified. For example a T-helper cell response (Th1/Th2) may be characterized by the measurement of IFN-γ and IL-4 secreting cells using by ELISA (e.g. BD Biosciences OptEIA kits). Peripheral blood mononuclear cells (PBMC) or splenocytes obtained from a subject may be cultured, and the supernatant analyzed. T lymphocytes may also be quantified by fluorescence-activated cell sorting (FACS), using marker specific fluorescent labels and methods as are known in the art.

A microneutralization assay may be conducted to characterize an immune response in a subject, see for example the methods of Rowe et al., 1973. Virus neutralization titers may be obtained several ways, including: 1) enumeration of lysis plaques (plaque assay) following crystal violet fixation/coloration of cells; 2) microscopic observation of cell lysis in culture; 3) ELISA and spectrophotometric detection of one or more viral proteins, e.g. the NP virus protein (correlation with virus infection of host cells).

As used herein, an “immunostimulatory composition” refers to a composition to stimulate an immune response in a subject. The immunostimulatory composition may elicit a humoral immune response and/or a cell mediated immune response. In embodiments of the invention, the immunostimulatory composition may be a vaccine composition, an antiviral composition, an antiviral therapeutic composition or an antiviral prophylactic composition.

An immunostimulatory composition may comprise one or more antigens and optionally an immune modulating substance, i.e. a substance that initiates, catalyzes and/or modulates an immune response to a particular antigen. In an embodiment of the invention, the immune modulating substance is an adjuvant. The term “modulate” or the like refer generally to an increase or decrease in a particular response or parameter, as determined by any of several assays generally known or used. In embodiments of the invention, an immunostimulatory composition comprising an antigen and an immune modulating substance may elicit a cell mediated immune response in a subject. A vaccine composition that comprises an immune modulating substance may be known as an enhanced vaccine composition. In an embodiment, the immunostimulatory composition is an influenza vaccine, optionally a commercially available vaccine, such as a seasonal influenza vaccine or a pandemic influenza vaccine, typically a split-virus influenza vaccine (such as Fluviral, Vaxigrip, or Fluzone). As used herein, an “antigen” refers generally to any substance that is able to bind to an antibody or a T cell receptor.

As used herein, an “adjuvant” refers to any substance or substances that stimulates the immune system to increase the response to a specific antigen or group of antigens.

The ability of an antigen to induce a response of the innate or adaptive immune system may be described as the “biological activity” of the antigen. An adjuvant may mediate, augment or stimulate the biological activity of an antigen. In some examples, the antigen may have very little or negligible biological activity in the absence of an adjuvant.

An adjuvant may have an antigenic effect that is independent of a specific antigen. For example, adjuvant compositions may induce maturation of some immune cells, or may induce clonal expansion of some immune cells, or may induce cytokine production in some immune cells. As used herein, “immune cells” refers to cells of the immune system, and include, without limitation, peripheral blood mononuclear cells (PBMC), granulocytes (CD 15+), monocytes, (CD 14+), T-lymphocytes (CD3+), T helper cells (CD4+), cytotoxic T cells (CD8+), B lymphocytes (CD 19+, CD20+), dendritic cells and natural killer cells (CD16+, CD56+).

Examples of adjuvants include, but are not limited to, polyIC, polyICLC, polyIC/R, aluminum hydroxide, alum, Alhydrogel™ (aluminum trihydrate) or other aluminum-comprising salts, virosomes, nucleic acid molecules comprising CpG motifs, squalene, oils, MF59, QS21, various saponins, virus-like particles, monophosphoryl-lipidA/trehalose dicorynomycolate, toll-like receptor agonists, copolymers such as polyoxypropylene and polyoxyethylene, or the like. Examples of adjuvants or adjuvant compositions are also described in, but are not limited to, PCT Publication WO 2009/086640, and U.S. Pat. Nos. 7,105,162, 7,148,191 and 6,869,607. Adjuvants may stimulate through Toll-like receptors (TLR); and such adjuvants include, without limitation, glucopyranosyl lipid adjuvant-stable emulsion (GLA-SE) (TLR4), resiquimod (TLR7), poly I:C (TLR3), and CpG (TLR9). In some embodiments an adjuvant that stimulates a Toll-like receptor may be preferable, and may elicit a cell-mediated immune response in the subject receiving same.

The activity of an adjuvant, an antigen, or a composition comprising an adjuvant and an antigen may be measured by assays known in the art. For example, induction of antigen-specific CD8-positive T lymphocytes may be quantified through use of an ELISPOT assay (Asai et al 2000 Clin. Diag. Lab Immunol 7:145-154). Other T-cell assays that may be useful for monitoring an immune response include intracellular cytokine flow cytometry, proliferation assays, antibody microarrays, and the like. See, for example Nagorsen et al 2004. [Expert Opin Biol Ther 4: 1677-84], or Handbook of Experimental Immunology, Vols. 1-IV, [D. M. Weir and C. C. Blackwell, eds., 1986, Blackwell Scientific Publications]. Antigen-specific antibodies may be detected and/or quantified using any of several assays known in the art. Examples include ELISA, western blot, flow-cytometry or bead-based methods such as RapidQuant™ (Guava Technologies) or the like. Antibodies may be of several isotypes or subtypes, such a's IgA, IgM, IgG, IgD and IgE, with particular isotypes or subtypes being predominant in certain tissues, in response to type of pathogens (bacterial, viral, parasite or protozoan) and/or at certain stages in the immune response.

Activity, or biological activity, including cytokine production, may be assessed with regards to the subject as a whole (e.g. via a serum, blood or other fluid or tissue sample), or with regards to cells, or a particular cell type. The cells may be, for example, peripheral blood mononuclear cells (PBMCs) or particular immune cells, such as CD8+ cells or CD4+ cells. Quantities and/or concentrations may be calculated on a mass/mass basis (e.g. micrograms or milligrams per kilogram of subject), or may be calculated on a mass/volume basis (e.g. concentration, micrograms or milligrams per milliliter).

An antigen may be prepared by methods known in the art. For example, an antigen may be prepared from a killed whole-organism (a ‘killed vaccine’) or may be prepared from a specific protein, peptide or other substructure of a pathogen. Alternatively, the antigen may be a fusion protein comprising a whole or partial protein or peptide from a pathogen, fused with another protein or peptide from another organism or with a non-pathogen protein or peptide, or a moiety to impart a quality to the fusion protein, e.g. a moiety that is useful in purification of the antigen, such as a His-Tag. An antigen may be soluble in an aqueous medium, or a lipophilic medium (e.g. an oil, fat or cream) or may comprise a suspension in an aqueous or lipophilic medium. Specific proteins or peptides may be produced using molecular biology techniques or methods (“recombinant” proteins or peptides). Conventional techniques or methods used in recombinant molecular biology are described in, for example, Molecular Manual 3^{rd} edition. Sambrook and Russell. CSHL Press, Cold Spring Harbour, New York; Current Protocols in Molecular Biology, 2007 Ausubel et al editors. Wiley InterScience, New York; Current Protocols in Immunology, 2006 Coligan et al editors. Wiley InterScience, New York. Recombinant antigens may be expressed using a recombinant expression system, for example bacterial, yeast, baculoviral, mammalian cell or plant expression system.

Examples of antigens include, but are not limited to, a nucleic acid, a protein, a peptide, a fusion protein, a fusion peptide, a recombinant protein or recombinant peptide or an amino acid chain of an antigen from a viral pathogen, or one or more than one fragments or portions thereof.

Examples of bacterial, fungal or viral pathogens, include, but are not limited to, papilloma, influenza, hepatitis A, hepatitis B, hepatitis C, hepatitis D, hepatitis E, hepatitis G, cytomegalovirus, Epstein Barr virus, varicella, Ebola, herpes simplex, herpes zoster, human papillomavirus (HPV), variola, Norwalk virus, rotavirus; or the causative agents of the following diseases or disorders; genital warts; AIDS; AIDS Related Complex; chickenpox; shingles, common cold; cytomegalovirus infection; Colorado tick fever; Dengue fever; haemorrhagic fever; hand, foot and mouth disease; hepatitis; Flu; Lassa fever; measles; Marburg haemorrhagic fever; infectious mononucleosis; mumps; poliomyelitis; progressive multifocal leukencephalopathy; rabies; Rubella; SARS; smallpox; viral encephalitis; viral gastroenteritis; viral meningitis; fifth disease; viral pneumonia; West Nile disease; and Yellow fever. Examples of influenza antigens are described in PCT Publication WO 2010/003225.

Immunostimulatory compositions according to various embodiments of the invention may be formulated with substances, and according to methods, known in the art. For example, an immunostimulatory composition may be formulated with a pharmaceutically acceptable carrier or excipients, and optionally in a vehicle such as, for example, an aqueous vehicle such as water, Ringer's lactate, isotonic saline or the like. Examples of pharmaceutically acceptable excipients include, without limitation, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, anti-adherents agents, disintegrants, coatings, glidants, deflocculating agents, anti-nucleating agents, surfactants, stabilizing agents, non-aqueous vehicles such as fixed oils, polymers or encapsulants for sustained or controlled release, ointment bases, fatty acids, cream bases, emollients, emulsifiers, thickeners, preservatives, solubilizing agents, humectants, water, alcohols or the like. See, for example, Berge et al. (1977. J. Pharm Sci. 66:1-19), or Remington—The Science and Practice of Pharmacy, 21^{st} edition. Gennaro et al editors. Lippincott Williams & Wilkins Philadelphia.

In another aspect, the invention relates to a method of determining a subject's ability to develop a cell-mediated immune response, the method comprising determining a level of baseline Granzyme B activity in a sample from a subject, wherein the level of baseline Granzyme B activity that is greater than a level of baseline Granzyme B activity of a chronic disease negative population at a 95% confidence interval is indicative of the subject's inability, or poor ability, to develop a cell-mediated immune response, especially compared to healthy older adult control subjects that receive an immunostimulatory composition.

In another aspect, the invention relates to a method of determining an immune response of a subject to an immunostimulatory composition, where the method comprises (a) immunizing the subject with the immunostimulatory composition, (b) determining a proportion of cells that express one or more biomarkers in a test sample that was obtained from the subject and stimulated with the immunostimulatory composition, (c) comparing the proportion of cells that express the one or more biomarkers in the test sample to a proportion of cells that express one or more biomarkers in a control sample, and (d) determining a strength of the immune response to the immunostimulatory composition by detecting a difference in the proportion of cells that express the one or more biomarkers in the test sample and the control sample. Optionally, in one embodiment, the test sample in step (b) may be stimulated with the target to which the immunostimulatory composition is directed against. For example, in one embodiment, the immunostimulatory composition is a flu vaccine, and the target is influenza virus.

In one embodiment, a difference in a proportion of cells that express the one or more biomarkers may be useful in determining a strength of the immune response to the immunostimulatory composition. The phrase a “proportion of cells” may, in one embodiment, refer to the portion of cells in the sample that expresses the one or more biomarkers. In another embodiment, the “proportion of cells” refers to the fraction of cells that expresses one biomarker in relation to the fraction of cells that express that one biomarker and another biomarker.

In certain embodiments, determination of a decrease in the proportion of GzmB+/Perf+ cells in the subset of cells that express CD8+/CD45RA+/CCR7−/GzmB+/Perf+; CD4+/CD45RA+/CCR7−/GzmB+/Perf+; CD8+/CD45RA+/CCR7−/GzmB+/Perf+/CD25+/CD127+; or CD4+/CD45RA+/CCR7−/GzmB+/Perf+/CD25+/CD127+ in the test sample compared to the control sample, by at least about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.2, 4.4, 4.6, 4.8, 5.0, 5.5, 6.0, 6.5, 7.0, 7.5, 8.0, 8.5, 9.0, 9.5, or 10 fold indicates development of a poor immune response in the subject to the immunostimulatory composition. In other embodiments, determination of a similar proportion of GzmB+/Perf+ cells in the subset of cells that express CD8+/CD45RA+/CCR7−/GzmB+/Perf+; CD4+/CD45RA+/CCR7−/GzmB+/Perf+; CD8+/CD45RA+/CCR7−/GzmB+/Perf+/CD25+/CD127+; or CD4+/CD45RA+/CCR7−/GzmB+/Perf+/CD25+/CD127+ in the test sample compared to the control sample, indicates development of a strong immune response in the subject to the immunostimulatory composition. In these respects, the control sample may represent samples from healthy young adults that have been immunized with the immunostimulatory composition. As used herein, a similar proportion of GzmB+/Perf+ cells in the CD4 or CD8 subsets refers to a difference of no greater than about 1%, 2%, 3%, 4%, 5%, 6%, 7%, 8%, 9%, 10%, 11%, 12%, 13%, 14%, 15%, 16%, 17%, 18%, 19%, or 20% in the test sample compared to the control sample.

As used herein, a “strength” of an immune response refers to the magnitude of an immune response. For example, an immunostimulatory composition may elicit a strong immune response in a subject, or a poor immune response. The terms strong and poor are described herein.

In a further embodiment, the method includes comparing biomarker profiles in samples taken from a subject at different time points. Accordingly, the methods described herein may be used to monitor the progression of immune response to immunization in a subject or group of subjects at different time points. In one embodiment, a test sample is taken from a subject pre-vaccination and subsequent samples are taken at periodic intervals of between 1 hour and 20 or more weeks post-vaccination. In one embodiment, test samples may be taken pre-vaccination, upon vaccination, and at 1 week, 4 weeks, 10 weeks and/or 20 weeks post vaccination. In one embodiment, the test samples are taken at any other suitable time interval for monitoring the subject. In another embodiment, control samples are taken from healthy young adults at the same intervals as the test sample.

In another aspect, the invention relates to a method for determining an immune response of a subject to an immunostimulatory composition, the method comprising: (a) determining a first level of granzyme B (bGrzB) activity from a first sample from the subject, (b) administering the immunostimulatory composition to the subject, (c) determining a second level of granzyme B (tGrzB) activity from a second sample from the subject, and (d) calculating an induced level of granzyme B (iGrzB) activity, where iGrzB activity=fold increase in tGrzB activity over bGrzB activity. A high level of bGrzB activity, a low level of iGrzB activity or a low level of tGrzB activity indicates that the subject is at risk of developing a poor immune response to the immunostimulatory composition, and a low level of bGrzB activity or a high level of iGrzB activity indicates that the subject is at risk of developing a strong immune response to the immunostimulatory composition.

In one embodiment, a poor immune response may be a cell-mediated equivalent of seroprotection as log iGrzB levels of <1.8, or seroconversion as not achieving the fold increase in iGrzB needed for a CMV+ subject (iGrzB=1.5 pre-vaccination) to have protective levels of iGrz (>2.0) following vaccination. In one embodiment, a strong immune response may be a cell-mediated equivalent of seroprotection as log iGrzB levels of >2.0, or seroconversion as the fold increase in iGrzB that would be needed for a CMV+ subject (iGrzB=1.5 pre-vaccination) to have protective levels of iGrz (>2.0) following vaccination.

In one embodiment, the invention relates to use, to determine a response of a subject to an immunostimulatory composition, of a level of granzyme B activity determined from a sample of the subject before (background granzyme B (bGrB)) and after (total granzyme B (tGrB)) administration of the immunostimulatory composition, wherein tGrB=bGrB+ induced granzyme B (iGrB), and wherein a low iGrB or a high bGrB is predictive of the subject having a poor response to the immunostimulatory composition, and wherein a high iGrB or a low bGrB is predictive of a subject having a good response to the immunostimulatory

In another embodiment, the invention relates to a method of assessing a response of a subject to an immunostimulatory composition comprising a determination of a subject's background Granzyme B (bGrB) activity; administering to the subject an immunostimulatory composition; determining the subject's Granzyme B activity following administration of the immunostimulatory composition (tGrzB); calculating an immunostimulatory composition-responsive Granzyme B (iGrB) activity; wherein a low iGrB or a high bGrB is predictive of the subject having a poor response to the immunostimulatory composition, and wherein a high iGrB or a low bGrB is predictive of a subject having a good response to the immunostimulatory composition.

For some subjects, GrB activity may only be determined after the subject has received an immunostimulatory composition. Thus, the subject's tGrB, if low, is predictive of the subject having a poor response to the immunostimulatory composition.

A low tGrB level may be less than about 990 U/mg, less than about 975 U/mg, less than about 950 U/mg, less than about 925 U/mg, less than about 900 U/mg, less than about 875 U/mg, less than about 850 U/mg, less than about 825 U/mg, less than about 800 U/mg, less than about 775 U/mg, less than about 750 U/mg, less than about 725 U/mg, less than about 700 U/mg, less than about 675 U/mg, less than about 650 U/mg, less than about 625 U/mg, less than about 600 U/mg, less than about 575 U/mg, less than about 550 U/mg, less than about 525 U/mg, less than about 500 U/mg, less than about 475 U/mg, less than about 450 U/mg, less than about 425 U/mg, less than about 400 U/mg, less than about 375 U/mg, less than about 350 U/mg, less than about 320 U/mg, or less than about 300 U/mg of granzyme B.

A high bGrB level may be greater than about 500 U/mg, greater than about 510 U/mg, greater than about 520 U/mg, greater than about 530 U/mg, greater than about 540 U/mg, greater than about 550 U/mg, greater than about 560 U/mg, greater than about 570 U/mg, greater than about 580 U/mg, greater than about 590 U/mg, greater than about 600 U/mg, greater than about 610 U/mg, greater than about 620 U/mg, greater than about 630 U/mg, greater than about 640 U/mg, greater than about 650 U/mg, greater than about 660 U/mg, greater than about 670 U/mg, greater than about 680 U/mg, greater than about 690 U/mg, greater than about 700 U/mg, or greater than about 725 U/mg of Granzyme B.

A high tGrB level may be greater than about 990 U/mg, greater than about 1000 U/mg, greater than about 1025 U/mg, greater than about 1050 U/mg, greater than about 1100 U/mg, greater than about 1125 U/mg, greater than about 1150 U/mg, greater than about 1175 U/mg, greater than about 1200 U/mg, greater than about 1300 U/mg, greater than about 1400 U/mg, or greater than about 1500 U/mg of Granzyme B.

A low bGrB may be less than about 300 U/mg, less than about 290 U/mg, less than about 280 U/mg, less than about 270 U/mg, less than about 260 U/mg, less than about 250 U/mg, less than about 240 U/mg, less than about 230 U/mg, less than about 220 U/mg, less than about 210 U/mg, less than about 200 U/mg, less than about 175 U/mg, less than about 150 U/mg, less than about 125 U/mg, less than about 100 U/mg, less than about 75 U/mg, less than about 50 U/mg, less than about 25 U/mg, less than about 10 U/mg, or about 0 U/mg of Granzyme B.

In various embodiments, a low iGrB level has a base 10 log iGrB value that is lower than about 1.8, 1.7, 1.6, 1.5, or lower; whereas a high iGrB level has a base 10 log iGrB value that is greater than 1.9, 2.0, 2.1, 2.2, 2.3, 2.4, 2.5 or greater.

A subject found to have a low iGrB level, a low tGrB level or high bGrB level may be further provided with an enhanced immunostimulatory composition having an adjuvant for stimulating a cell mediated immune response. Alternatively, or in addition, the subject may receive one or more therapeutic agents, such as antiviral medicaments, or receive or participate in one or more treatment regimens that may reduce or ameliorate the subject's inability to develop a strong immune response, and thus, for example, reduce or ameliorate the subject's risk of developing a viral infection.

In a further aspect, the invention relates to a method for determining an immune response of a subject to an immunostimulatory composition, the method comprising: (a) immunizing the subject with the immunostimulatory composition, (b) determining a first level of granzyme B (bGrzB) activity in a test sample from the subject, (c) stimulating the test sample with the immunostimulatory composition, (d) determining a second level of granzyme B (tGrzB) activity from the test sample, (e) calculating an induced level of granzyme B (iGrzB) activity, wherein iGrzB activity is equal to fold increase in tGrzB activity over bGrzB activity, and (f) comparing the iGrzB activity in the test sample to an iGrzB activity of a control sample, wherein a decrease in the level of bGrzB activity and/or an increase in the level of iGrzB activity in the test sample compared to the control sample indicates development of a strong immune response in the subject to the immunostimulatory composition, and wherein a similar or an increase in the level of bGrzB activity and/or a similar or a decrease in the level iGrzB activity in the test sample compared to the control sample indicates development of a poor immune response in the subject to the immunostimulatory composition. Optionally, in one embodiment, the test sample in step (c) may be stimulated with the target to which the immunostimulatory composition is directed against. For example, in one embodiment, the immunostimulatory composition is a flu vaccine, and the target is influenza virus.

In one embodiment, the level of bGrzB activity in the test sample compared to the control sample that is indicative of development of a strong immune response in the subject to the immunostimulatory composition is decreased by at least about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 fold, optionally, 4 fold. In one embodiment, the level of iGrzB activity in the test sample compared to the control sample that is indicative of development of a strong immune response in the subject to the immunostimulatory composition is increased by at least about 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 fold, optionally, 4 fold. In these regards, the control sample represents subjects who are chronic disease positive, typically who are also older adults.

In various aspects, the invention relates to methods of treating a subject. In one aspect, the invention relates to a method of treating a subject in need thereof, the method comprising (a) identifying the subject determined to develop a poor immune response to an immunostimulatory composition according to a method of the invention, and (b) treating the subject. In another aspect, the invention relates to a method of treating a subject in need thereof by (a) determining a level of granzyme B activity in a test sample from the subject, and (b) if the level of granzyme B activity in the sample from the subject is greater than an average level of granzyme B activity in samples from a chronic disease negative population, then (i) treating the subject.

In one embodiment, a subject determined to develop a poor immune response to the immunostimulatory composition, or a subject with a level of granzyme B activity greater than an average level of granzyme B activity in samples from a chronic disease negative population, may be treated. For example, in one embodiment, the subject may be treated with the immunostimulatory composition according to an altered immunization schedule. An altered immunization schedule may be based on the standard of care for the immunostimulatory composition and/or the pathogen or condition to which the immunostimulatory composition is directed. For example, the altered immunization schedule may be no more than 1 week, no more than 2 weeks, no more than 3 weeks, no more than 4 weeks, no more than 5 weeks, no more than 6 weeks, no more than 7 weeks, no more than 8 weeks, no more than 9 weeks before a specific time, e.g. an annually recurring time period characterized by the prevalence of outbreaks of the pathogen to which the immunostimulatory composition is directed, e.g. seasonal flu. In another embodiment, the subject may be treated with an enhanced immunostimulatory composition. As used herein, an “enhanced immunostimulatory composition” comprises the immunostimulatory composition and one or more immune modulating substances, e.g. an adjuvant. An enhanced immunostimulatory composition may be useful in eliciting a response of a specific aspect of an immune response, for example, a cell mediated immune response. In another embodiment, the subject may be treated with a therapeutic agent. In a further embodiment, the subject may be treated with the immunostimulatory composition according to an altered immunization schedule and/or an enhanced immunostimulatory composition; and optionally with a therapeutic agent. For example, the therapeutic agent may be an antiviral agent.

In one embodiment, a subject is treated if a test sample derived therefrom has a level of baseline granzyme B activity that is greater than about 300 U/mg, greater than about 325 U/mg, greater than about 350 U/mg, greater than about 375 U/mg, greater than about 400 U/mg, greater than about 425 U/mg, greater than about 450 U/mg, greater than about 475 U/mg, greater than about 500 U/mg, greater than about 525 U/mg, greater than about 550 U/mg, greater than about 575 U/mg or greater than about 600 U/mg compared to an average level of granzyme B activity in samples from a chronic disease negative population. In another embodiment, a subject is treated if the level of baseline granzyme B activity in the test sample is at least 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 fold greater compared to the average level of baseline granzyme B activity in samples from a chronic disease negative population.

In another aspect, the invention relates to a method of increasing treatment efficiency for a viral infection, the method comprising: determining a risk profile of a subject by determining a level of granzyme B activity in the subject, wherein the subject has an increased risk profile if the level of granzyme B activity in the sample is greater than a level of granzyme B activity in a chronic disease negative population, and administering a therapeutic agent to the subject with the increased risk profile prior to the subject developing a symptom of a viral infection.

In an embodiment of the invention, the method of increasing treatment efficacy is for a viral infection wherein a subject having an increased immune risk profile is administered an anti-febrile and or an anti-viral drug in advance of development of one or more symptoms of a viral infection.

In embodiments of the invention, a level of baseline granzyme B activity greater than about 300 U/mg, greater than about 325 U/mg, greater than about 350 U/mg, greater than about 375 U/mg, greater than about 400 U/mg, greater than about 425 U/mg, greater than about 450 U/mg, greater than about 475 U/mg, greater than about 500 U/mg, greater than about 525 U/mg, greater than about 550 U/mg, greater than about 575 U/mg or greater than about 600 U/mg in a sample from the subject indicates a subject having an increased immune risk profile. In another embodiment, a subject has an increased immune risk profile if the level of granzyme B activity in the sample is at least 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 fold greater compared to a level of granzyme B activity in a chronic disease negative population.

As used herein, an “immune risk profile” refers to an indicator of the likelihood of a subject to develop a protective immune response when administered an immunostimulatory composition. In embodiments of the invention, an increased immune risk profile is observed in a subject having one or more of the following characteristics: CMV positive, bGrB greater than about 300 U/mg protein in a sample, bGrB greater than the bGrB of a CMV negative population, older age (greater than 60 years old), or frailty. The frailty of a subject may be defined by accumulation of deficits, and scored by a Frailty Index (FI) (reviewed in Rockwood 2011).

A subject, or a sample from a subject, may be tested for GrB levels or GrB activity, or both GrB level and activity in part to obtain the subject's risk profile and/or determine the subject's risk of a poor response to an immunostimulatory composition, or risk of severity of infection from a viral infection, either in the presence or absence of vaccination. To evaluate a subject's risk, a sample or samples may be collected from the subject, mixed with an anticoagulant, and the sample centrifuged to separate the blood into plasma, red cells and white cell (buffy coat) fractions. The separated fractions may be subsequently analyzed for GrB level and/or activity or stored for later analysis.

In the context of the present invention, the terms “treatment”, “treating”, “therapeutic use” or “treatment regimen” as used herein may be used interchangeably to encompass, without limitation, prophylactic, palliative, and therapeutic modalities of administration of the compositions of the present invention, and include any and all uses of the described compositions that remedy or prevent a disease state, condition, symptom, sign, or disorder caused by an infection, or reduce the severity of a disease state, condition, symptom, sign or disorder caused by an infection. Thus, any prevention, amelioration, alleviation, reversal, or complete elimination of an undesirable disease state, symptom, condition, sign, or disorder associated with an inflammation-based pathology, or other disease or disorder that benefits from stimulation of the body's immune response, is encompassed by the present invention. A treatment may comprise administration of an effective amount of an antigen, alone or in combination with an adjuvant, and/or in combination with a therapeutic agent or regimen.

An “effective amount” of an adjuvant as used herein refers to the amount of adjuvant required to have an immunostimulatory effect when co-administered with an antigenantigen. The effective amount may be calculated based on known principles, for example, on a mass/mass basis (e.g. micrograms or milligrams per kilogram of subject), or may be calculated on a mass/volume basis (e.g. concentration, micrograms or milligrams per milliliter). Using a mass/volume unit, an antigen may be present at an amount from about 0.1 μg/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 μg/ml, or any amount therebetween; or from about 1 μg/ml to about 2000 μg/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, μg/ml or any amount therebetween; or from about 10 μg/ml to about 1000 μg/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 μg/ml, or any amount therebetween; or from about 30 μg/ml to about 1000 μg/ml or any amount therebetween, for example 30.0, 35.0, 40.0, 50.0, 60.0, 70.0, 80.0, 90.0, 100, 120, 140, 160, 180, 200, 250, 500, 750, 1000 μg/ml.

Immunostimulatory compositions according to various embodiments of the invention may be administered or used by any of several routes, including, for example, subcutaneously, intraperitoneally, intramuscularly, intravenously, epidermally, transdermally, mucosal membrane administration, orally, nasally, rectally, topically or vaginally. Alternately, such compositions may be directly administered, used or injected directly into or tangentially to a tissue, e.g. an infected tissue, a tumor, or a lymph node near an infected tissue or a tumor, or into an organ or tissue near a tumor or infected tissue, or an organ or tissue comprising tumor cells or infected cells. See, for example, Remington—The Science and Practice of Pharmacy, 21^{st} edition. Gennaro et al editors. Lippincott Williams & Wilkins Philadelphia. Carrier formulations may be selected or modified according to the route of administration.

Immunostimulatory compositions may be may be administered to a subject in accordance with standard dosing regimens. For example, an immunostimulatory composition may be administered or used in a single-dose, or in several doses over time (e g minutes, hours, days, months or years). Dosage schedules may be dependent on a number of factors, for example, the subject's condition, age, gender, weight, route of administration, formulation, or general health. Dosage schedules may be calculated based on a number of factors, including, measurements of adsorption, distribution, metabolism, excretion and toxicity in a subject, or may be extrapolated from measurements on an experimental animal, such as a rat or mouse, for use in a human subject. Optimization of dosage and treatment regimens are known in the art and are discussed in, for example, Goodman & Gilman's The Pharmacological Basis of Therapeutics 11^{th} edition. 2006. LL Brunton, editor. McGraw-Hill, New York, or Remington—The Science and Practice of Pharmacy, 21^{st} edition. Gennaro et al editors. Lippincott Williams & Wilkins Philadelphia.

Various delivery systems may be suitable for administration or use of an antigen, antigen, adjuvant or immunostimulatory composition of the invention. For example, antigen, antigen, adjuvant or immunostimulatory composition may be encompassed in liposomes. Moreover, a site of administration may be ‘primed’ with an adjuvant composition according to various embodiments of the invention, followed by administration of an antigen. The adjuvant composition may comprise an antigen, or may lack a specific antigen. Other excipients that stabilize or otherwise enhance the immunostimulatory effect of the adjuvant and/or antigen may also be included in the adjuvant composition.

In another aspect, the invention relates to methods of selecting a subject to receive an enhanced immunostimulatory composition, by determining a level of baseline granzyme B activity in a sample from a subject, wherein the subject having a level of baseline granzyme B activity greater than a level of baseline granzyme activity in a chronic disease negative population is selected to receive the enhanced immunostimulatory composition.

In an embodiment of the invention, a subject having a level of baseline granzyme B activity greater than 300 U/mg protein is selected to receive an enhanced immunostimulatory composition.

In an embodiment of the invention, a subject is selected to receive an antiviral prophylactic composition, by a method comprising the determination of granzyme B activity in a sample from the subject, wherein the subject having baseline granzyme B activity greater than 300 U/mg protein is selected to receive the antiviral prophylactic composition. In some aspects, the antiviral prophylactic composition may comprise administration of an antiviral agent.

In an embodiment of the invention, a subject is selected to receive an enhanced vaccine composition, by a method comprising the determination of CMV serologic status of the subject, wherein the subject having a positive CMV serology is selected to receive the enhanced vaccine composition.

In an embodiment of the invention, a subject is selected to receive an antiviral prophylactic composition, by a method comprising determining a CMV serology status of the subject, wherein the subject having a positive CMV serology is selected to receive the antiviral prophylactic composition.

In an embodiment of the invention, a subject is selected to receive an immunostimulatory composition, by a method comprising the determination of a level of granzyme B in the subject; wherein the level of granzyme B in the subject greater than that of a CMV-negative population is indicative of a need for the subject to receive an immunostimulatory composition for eliciting a cell mediated immune response. The level of baseline granzyme B activity may be about 300 U/mg protein in the sample, or greater.

In an embodiment of the invention, a subject is selected to receive an immunostimulatory composition, by a method comprising the determination of a level of granzyme B in the subject; wherein the level of baseline granzyme B in the subject greater than about 300 U/mg protein in a sample from the subject indicates a need for the subject to receive an immunostimulatory composition for eliciting a cell mediated immune response.

As used herein, a “therapeutic agent” refers to, without limitation, a drug, immune stimulating composition or other agent administered to, or for use in, a subject to treat, prevent or ameliorate one or more symptoms of an infection, for example a viral infection. Therapeutic agents may include antiviral medicaments, antifebrile medicaments, or other agents that may be administered to, or is for use in, a subject infected with a virus. Examples of therapeutic agents include, without limitation, oseltamivir and zanamivir, or the like. As used herein, a “prophylactic agent” refers to a drug, immune stimulating composition or other agent administered to a subject to prevent one or more symptoms of an infection, for example as a result of a viral infection, or to prevent an infection, for example, a viral infection.

Assays and kits to perform methods of the invention are contemplated. In one embodiment, the kit comprises reagents to detect one or more biomarkers useful in determining an immune response in a subject to an immunostimulatory composition. For example, a kit may comprise reagents to detect and measure levels of granzyme B (e.g. mRNA or protein), granzyme B activity, and/or CMV (e.g. an antibody to CMV). In another embodiment, the kit comprises reagents to detect one or more biomarkers useful in monitoring an immune response in a subject to an immunostimulatory composition. For example, a kit may comprise reagents to detect and measure levels of granzyme B (e.g. mRNA or protein) granzyme B activity, perforin, CD4, CD8, CD45RA, CCR7, CD25 and/or CD127. A kit may optionally further comprise one or more reagents, buffers, packaging materials, instructions for using the kit and containers for holding the components of the kit.

Methods of the invention may have research, medical and industrial applications. Representative, non-limiting applications of the invention may include the detection, quantification and/or diagnosis of a subject's ability to develop a cell mediated immune response. This may be useful to i) assess risk of severe infection at the point-of-care in clinical decision-making as it relates to which vaccine would be most effective, whether seasonal prophylaxis should be used as an adjunct or an alternative to vaccination (where vaccination is contraindicated such as due to an allergy), or when antivirals should be initiated in acute respiratory illness, even in advance of a confirmed diagnosis to reduce the risk for acute respiratory failure; ii) predict the efficacy of a vaccine or enhanced efficacy of a new vaccine either in clinical trials or in the post-marketing phase of vaccine development, or iii) as a correlate of risk for progression of age related disorders such as chronic lung and heart disease, wherein the potential for terminally differentiated T cells to be attracted to sites of chronic inflammation (i.e., atherosclerotic plaques) or acute inflammation (influenza infection), and release granzyme B into the extracellular space causing tissue injury, exacerbating inflammatory responses, and contributing to increased frailty due to the multi-organ effects of systemic inflammation.

Embodiments of the present invention will be described with reference to the following Examples that are provided for illustrative purposes only and should not be used to construe or limit the scope of the invention

### EXAMPLES

The present invention is based, in part, on the surprising discovery and elucidation of the age-related changes in the cytolytic T cell response to influenza virus within the memory and effector subsets of CD4+ and CD8+ T cells. Indeed, it has been demonstrated that, in older adults, cytolytic activity against influenza-infected targets is severely compromised in CD8+ T cells but preserved in CD4+ T cell subsets when compared to young adults. Further, some influenza subtypes (e.g. H3N2) elicit a greater cytolytic response than others (e.g. pH1N1) in all age groups studied, and this is consistent with the observed increase in serious complication rates of pH1N1 infection. The invention is based, in part, on the compensatory response of CD4+ T cells to influenza as a mechanism of protection against influenza illness when CD8+ T cells become dysfunctional in older adults.

Granzyme B activity in influenza-activated PBMC correlates with protection against influenza illness in older adults, however it has also been demonstrated that a subset of CD8+ T cells express GzmB and CD107a in association with increased levels of GzmB activity in resting PBMC from older adults—this was not seen in young adults.

The results described herein indicate that, with aging, there is relative preservation of cytolytic function in CD4+ T cells responding to influenza, while the initial CD8+ T cell response to vaccination rapidly declines by 10-weeks post-vaccination.

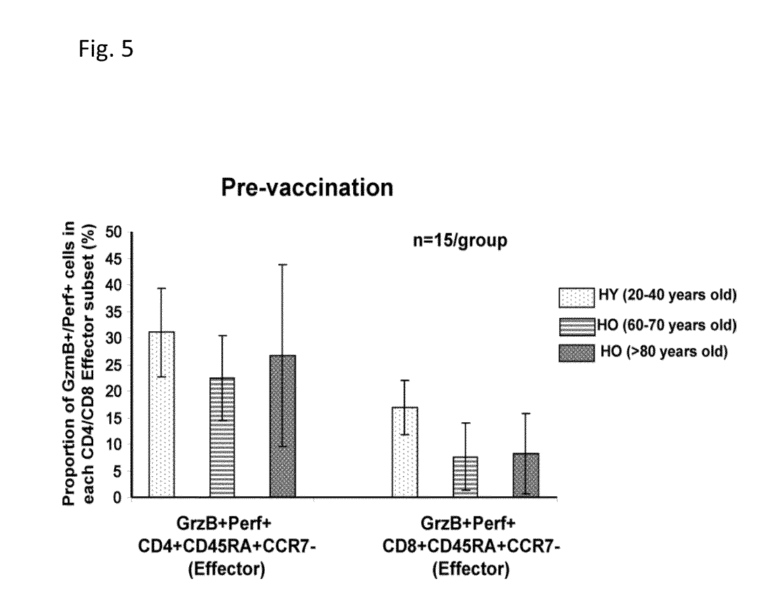

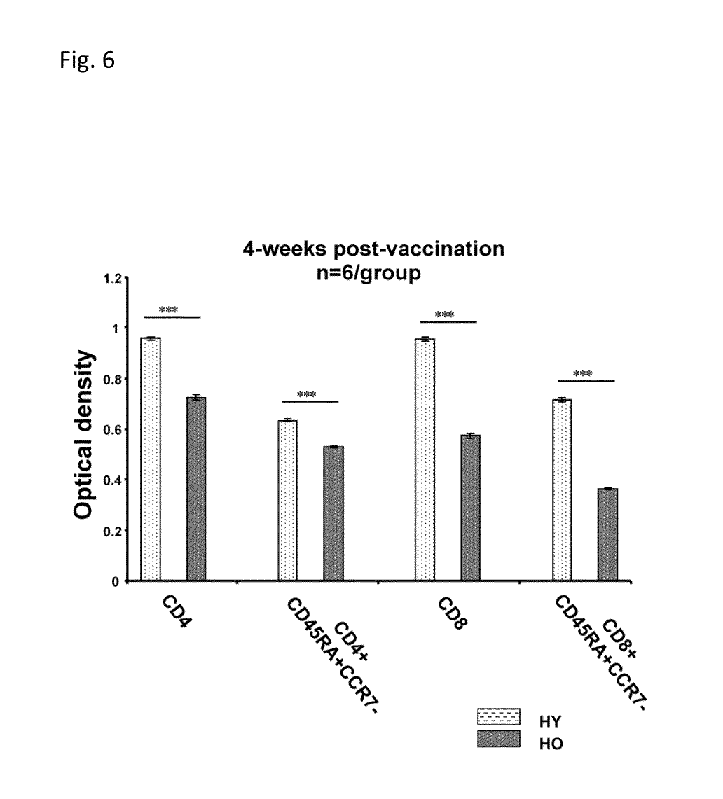

CD4+ effector T-cell subsets in older adults share phenotypic and functional characteristics with CD4+ and CD8+ effector T-cell subsets in young adults, and as such have an important role on controlling influenza infection. In some examples, this role may occur in the relative absence of functional cytotoxic CD8+ T cells in older adults. In addition, a diminished proliferative response to influenza virus stimulation in older compared to young adults was also demonstrated in these effector memory and effector CD8+ T cell subsets, while similar proliferative responses were found in these CD4+ T cells subsets (FIGS. 5 and 6).

The identification of influenza virus-specific CTL (GzmB+Perf+) using the degranulating marker CD107a, unexpectedly yielded a large proportion of GzmB+CD8+ T cells that expressed CD107a in older adults. Functional assays confirmed the reduction in cytolytic activity in CD8+ T cells responding to influenza challenge. These results show that the cytolytic potential on a per cell basis is reduced in influenza-specific CD8+ T cells of healthy older adults. In addition, a significant fraction of these CD8+ T cells are approaching a terminally differentiated state—it has been previously demonstrated that CD45RA+GzmB+CD8+ T cells mount a poor response to influenza virus [9].

A portion of the CD45RA+CD8+ T cells that accumulate with aging are due to the T cell response to chronic viral infections such as CMV [1]. With continuous exposure to the virus, these CMV-specific T cells are driven to become terminally differentiated T cells, preferentially exhausting the CD8+ T cell compartment and leading to immune compromise in older adults. While IL-7R alpha is expressed on T cells responding to acute viral infections such as influenza, it is not expressed on most CMV-specific T cells [23, 33].

GzmB+Perf+ effector T cells responding to influenza virus, expressed IL-7R alpha (CD127)—the dual expression of these cytolytic mediators therefore are employed to distinguish between terminally differentiated T cells and those T cells that could mount an effective influenza-specific cytolytic response to virus challenge. It should be noted that CD127+CD45RA+GzmB+Perf+ T cells are the phenotype of effector T cells in PBMC [24], which is maintained over the five days of in vitro stimulation and is the phenotype of T cell subset with demonstrated cytolytic activity in the experiments. This phenotype contrasts with the phenotype of CTL in the lungs, which have been activated by influenza-infected lung epithelial cells [23, 24].

To determine a mechanism for increased susceptibility to serious complications of influenza infection, the phenotype of cytolytic T cells was identified. The effector T-cell subset (GzmB+Perf+CD45RA+CCR7-CD127+) that expressed IL-2R (CD25) had high proliferative capacity and effective virus-specific cytolytic function in healthy young adults. In contrast, the CD25− effector subset exhibited limited proliferation and diminished cytolytic function in response to influenza virus. The proliferative response that yielded IL-2R+ effector T cells following in vitro virus stimulation was dramatically reduced in both CD4+ and CD8+ T cells in older compared to young adults. This result is consistent with the previously identified age-related decline in the in vitro proliferative response, which is IL-2 mediated [35]. Without wishing to be bound by theory, differences in the serious complication rates of pH1N1 relative to A/H3N2 infection in young adults, and A/H3N2 and pH1N1 infection in older relative to young adults, may be explained by a reduction in both the proliferative and cytolytic response to these influenza subtypes. Split virus vaccine compositions may be limited in their ability to stimulate cross reactive CTL responses—if such responses could be effectively stimulated in older adults, heterologous protection against different influenza subtypes and strains may be realized.

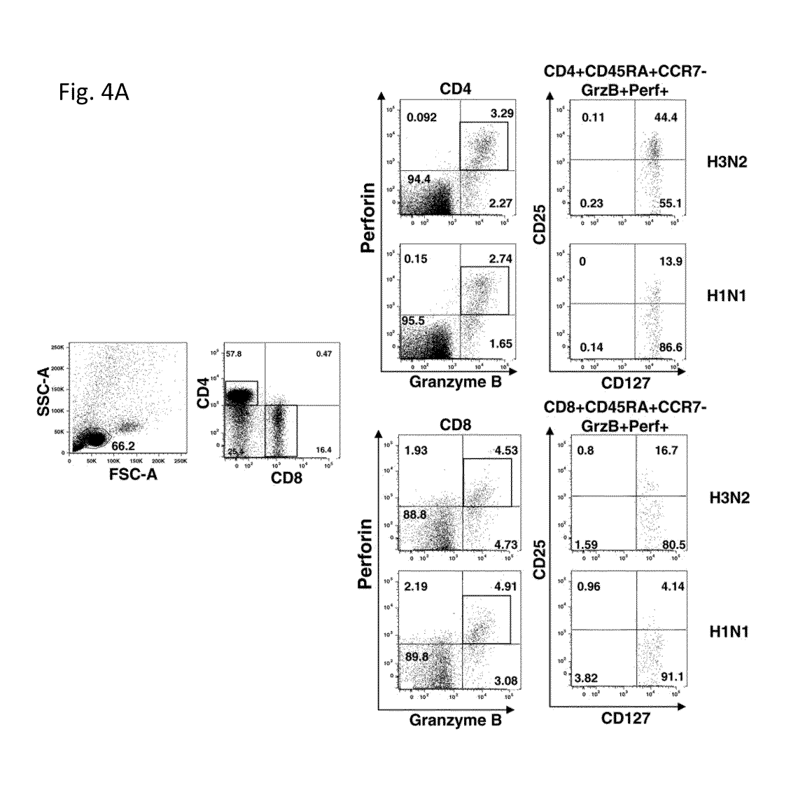

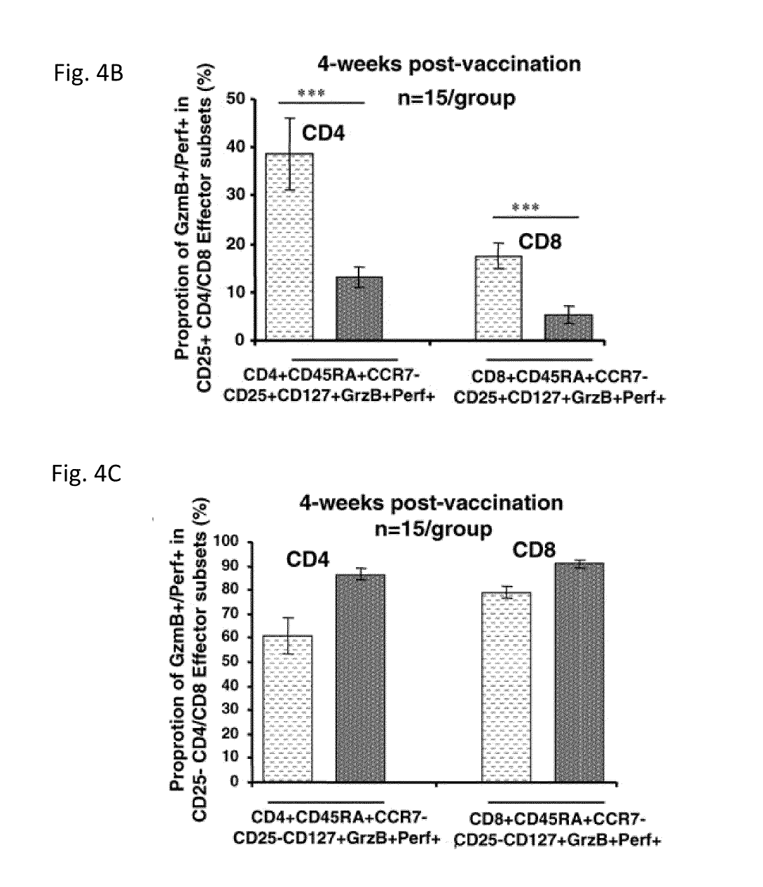

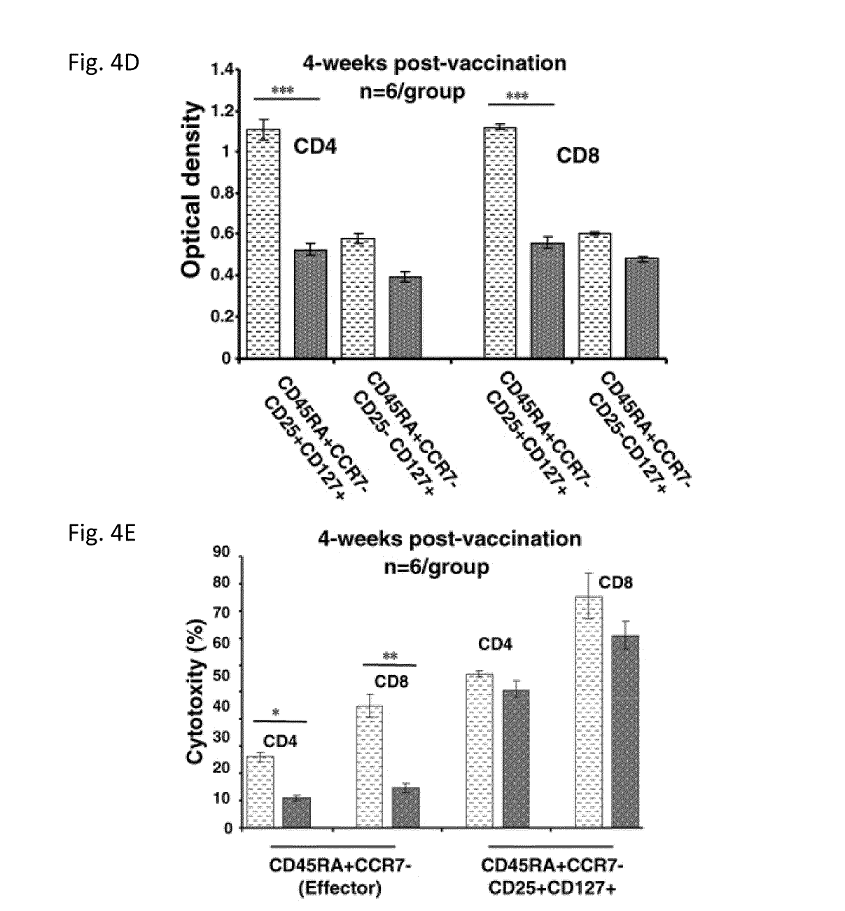

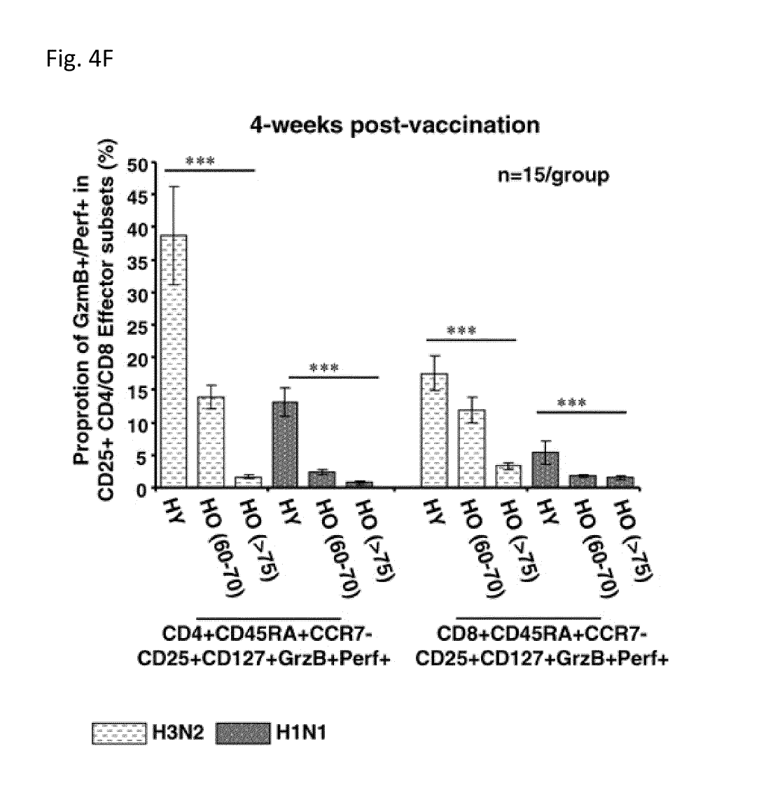

It is known that lower H1N1 attack rates in older compared to young adults can be attributed to sterilizing immunity provided by pre-existing antibody titers. These antibodies may also contribute to a reduction in illness severity when infection occurs in older adults. In contrast, young adults have lower antibody titers to H1N1 but demonstrate a higher CD4+ and CD8+ effector T cell response to the virus following vaccination with the seasonal H1N1 strain, as illustrated in FIG. 4. Protective antibody titers against pH1N1 were mainly observed in those adults over age 80 years old and this was consistent with very low attack rates in this subset of older adults.

In light of the rapid loss of CD8+ T cell memory, CD4+ T cell subsets are identified as a target cell population for stimulation of cell mediated immunity in immunostimulatory compositions for amelioration or prevention of viral infection, for example influenza.

Immunostimulatory compositions that stimulate cell mediated immune responses involving CD4+ T cells may comprise a higher dose of antigen, thus delivering more CD4 epitopes for antigen presentation. Alternately, adjuvants that more effectively stimulate presentation of both CD4 and CD8 epitopes may induce an enhanced response in both T cell subsets.

The immune cells of older adults may be capable of mounting an effective GzmB response to infection, and this response may be re-stimulated upon influenza vaccination after infection (e.g. for the next ‘flu season’, about 8-10 months later). An alternate embodiment of a suitable immunostimulatory composition may comprise an agent that stimulates GzmB expression, activation or expression and activation, or may comprise an agent that stimulates perforin expression, activation or expression and activation.

### Materials and Methods

### 1. Study Design and Participants

Fifteen young (20-25 years old (yo)) and older adults (60-70 years old (yo) or >80 years old (yo)) from the vicinity of the Greater Hartford Area of Connecticut were studied pre-vaccination and 4-weeks and 10-weeks post-vaccination in the fall and winter of 2008-2009. All subjects were recruited through written informed consent. The Institutional Review Board of the University of Connecticut Health Center approved the protocol and informed consent document. Subjects were excluded for an acute respiratory illness in the 2 weeks preceding study enrolment, insulin-requiring diabetes, any conditions or medication causing immunosuppression such as prednisone >10 mg/day, or any contraindications to influenza vaccination.

### 2. Cell Culture and Virus Stimulation

Human PBMC were isolated from venous blood samples by Histopaque gradient purification and stimulated in 0.5 ml of AIM V media (GIBCO) containing 1.0×106 PBMC and 4×10⁶ TCID₅₀ of influenza A/Victoria/3/75 (H3N2) virus as previously described [7] or stimulated with pandemic influenza A/TN/1-560-09 (pH1N1) virus (Generous gift from Dr. Richard J. Webby's lab, St. Jude Children's Research Hospital, Memphis). For proliferation and/or cytotoxicity assays, cells were stimulated with influenza virus 5 days or 5 μg/ml phytohemagglutinin (PHA) for 6 hr and cultured in AIM V medium containing 10% Human AB serum (Sigma) (R&D Systems).

### 3. Flow Cytometry and Antibodies

To determine the phenotype of T cells responding to influenza challenge, PBMC were prepared and stained as previously described [8]. Briefly, human anti-CD107a-PE (H4A3) was added to the PBMC/virus cultures, incubated for 12 hours, then the cells were washed with cold 0.2% FBS/PBS twice before adding the following conjugated monoclonal antibodies: Human anti-CD8-FITC (OKT8) and anti-CD25-APC-Cy7 (eBioscience), anti-CD127-Biotin/PE-Texas Red and anti-CCR7 PE-Cy7 (3D12) (BD Pharmingen), anti-CD4 Alex Fluor 700 (OKT4) and anti-CD45RA Pacific Blue/or eFluor 450 (HI100) (eBioscience). For intracellular staining, cells were fixed with 2% paraformaldyhyde and permeabilized with permeabilization buffer (eBioscience), then incubated with anti-GzmB Alex Fluor 647 (GB11) or anti-Perforin PE (δG9) (BD Pharmingen) antibodies. For cell sorting, human PBMC were prepared as above and sorted by using FACS Vantage SE/DIVA High Speed Sorter (BD Biosciences) running DIVA 5.0 Software. Data were acquired on the BD LSR II, and analysis using FlowJo 8.8.2 software.

### 4. Cytotoxicity Assay

Human PBMC were prepared and cultured in AIM V medium containing 10% human AB serum (Sigma) with influenza A/Victoria/3/75 or A/TN/1-560-09 (pH1N1) virus stimulation for 5-6 days. CD4+ and CD8+ T cell were selected by bead separation (Dyna Beads) and sorted into the different memory/effector subsets before the assay. PHA-activated autologous lymphoblast target cells were infected and cultured with influenza H3N2 or pH1N1 virus for 30 min and added into V-bottom 96-well plate at the dilution of 104 cells/100 μl per well in triplicate before adding sorted effector T-cell subsets at E/T (effector/target) ratios of 10:1 and 5:1; co-cultures were incubated for 4 hr at 37′C [14, 15]. The lactate dehydrogenase (LDH) activity released in the cell free supernatants was determined using the CytoTox 96® Non-Radioactive Cytotoxicity Assay (Promega, Madison, Wis.).

### 5. Proliferation Assay

The proliferative response to influenza was assessed by the MTT Cell Proliferation Assay (ATCC), based on the reduction of MTT (3-(4,5-dimethylthiazolyl-2)-2,5-diphenyltetrazolium bromide) by mitochondrial dehydrogenase of intact cells to formazan (MTT metabolic product). Briefly, human PBMC were stimulated with influenza virus for 5-6 days in AIM V medium containing 10% human AB serum. Sorted CD4+ and CD8+ T cell subsets were added to a flat bottom 96-well microtiter plate (1×105 cells/100 μl per well) with 10 μl of MTT reagent, incubated for 3-4 hr, and then 100 μl of Detergent Reagent added and incubated in the dark for 4 hr at RT. The amount of formazan produced (A570) was used to quantify the proliferative response.

### 6. Statistical Analysis

Unpaired t tests were used to compare the two groups for the different CD4+ and CD8+ T-cell subsets. Analyses were performed using Origin Pro 8.0 (OriginLab, MA, www.OriginLab.com). P values <0.05 were considered statistically significant.

For initial exploratory data analysis, a general linear model (GLM) was used to determine the relationships between GrzB levels, and health status, CMV status, change in levels in response to vaccination, and the development or not of LDI and for confounders in the analysis. Analyses were performed using SPSS 12.0 (SPSS Inc., Chicago, Ill.). Analysis of variance (ANOVA) and regression models were used to compare Grz B levels within and across different time points in the study.

A threshold level of protection was defined as a GrzB level greater than the upper limit of the 95% confidence interval for the Flu subset at 4-weeks post-vaccination.

### EXAMPLES

### Example 1

### A Phenotype Shift in T Cell Subsets in Vaccinated Older Compared to Young Adults

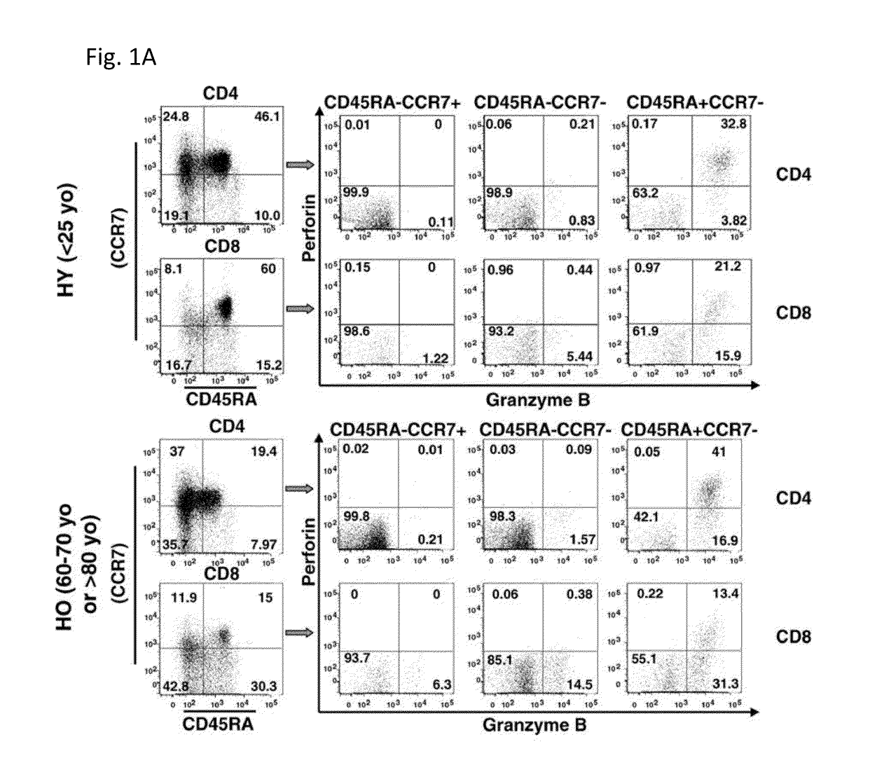

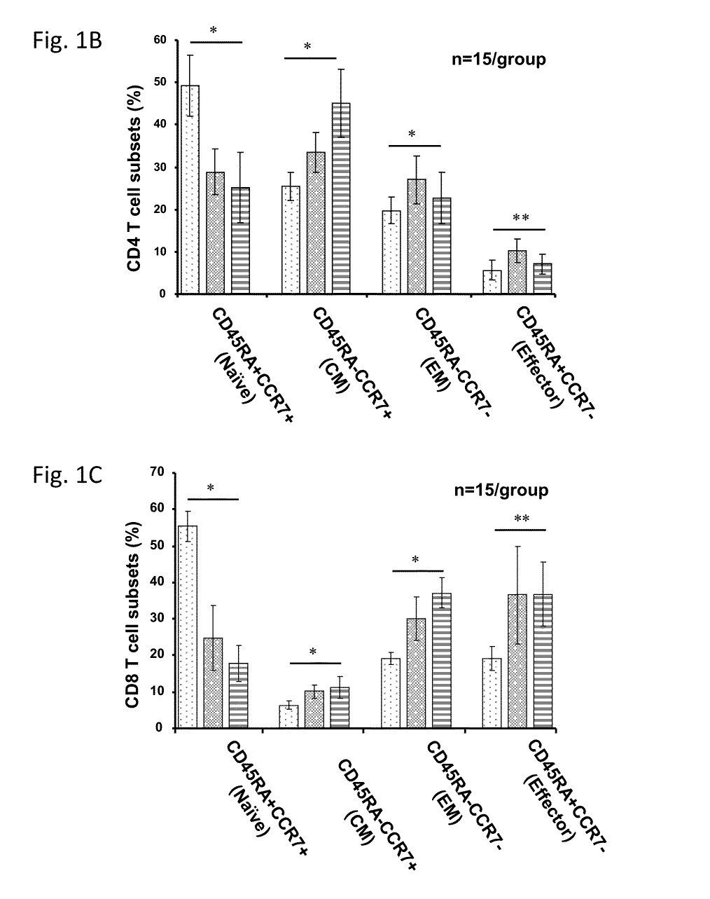

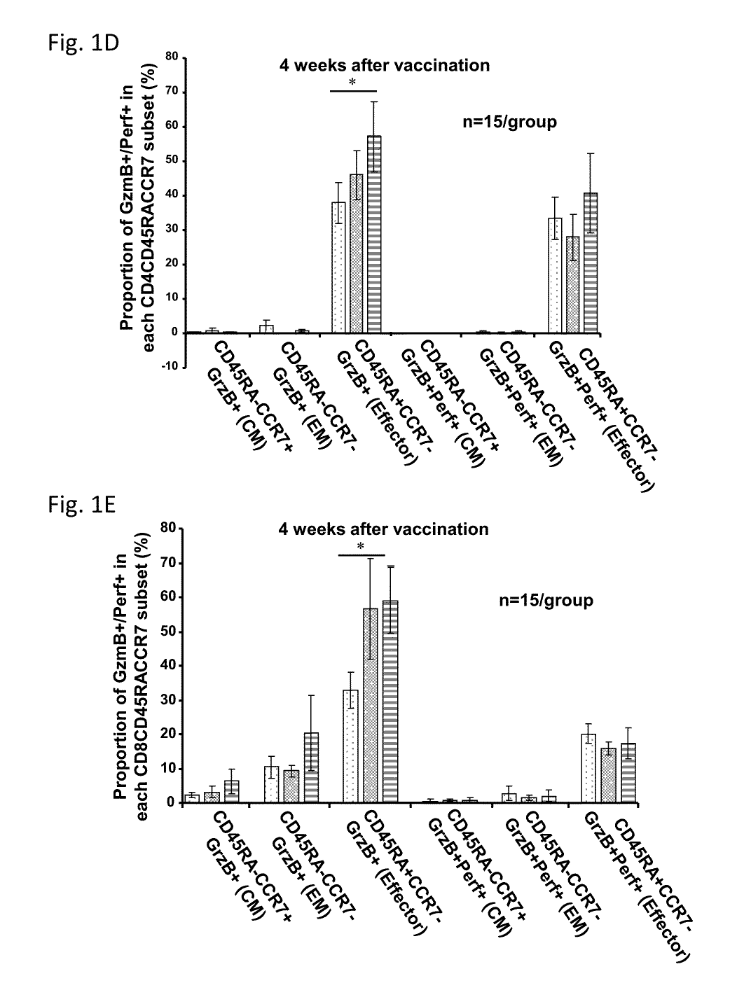

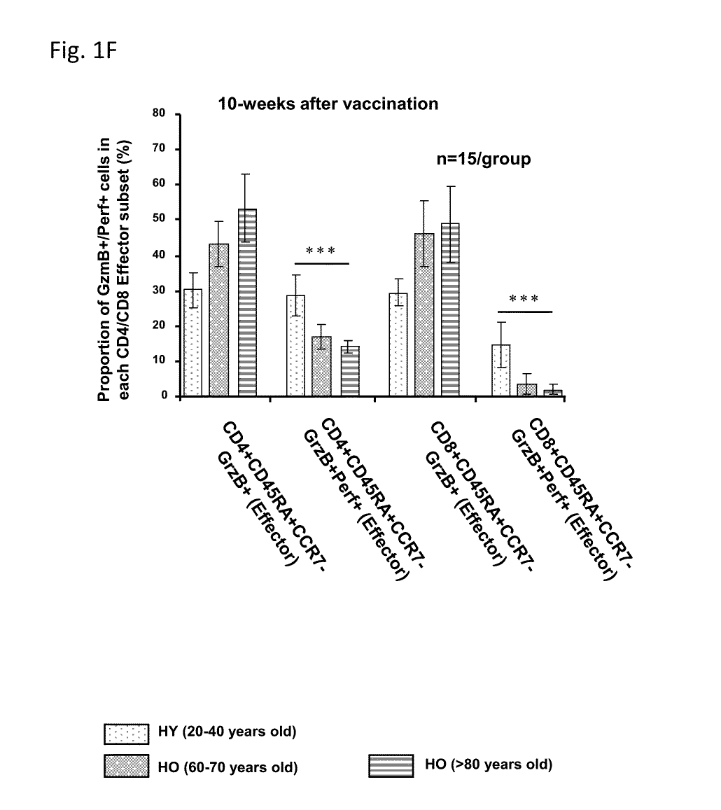

To determine the effect of age on the phenotype of naïve, memory and effector T lymphocytes responding to influenza challenge, PBMC from three age groups: 20-25 years old (yo), 60-70 yo, and >80 yo, were stimulated for 20 hours with live influenza A/H3N2 virus and analyzed by flow cytometry according to CD45RA+CCR7+ (naïve), CD45RA-CCR7+(central memory), CD45RA−CCR7− (effector memory) and CD45RA+CCR7− (effector) subsets (FIG. 1A) [16]. Consistent with contraction of the peripheral naïve T cell pool and the effect of aging on CD4+ and CD8+ T cells [17], there was a significant decline with age in the proportion of naïve (CD45RA+CCR7+) CD4+ and CD8+ T cells (FIGS. 1A, 1B and 1C, p<0.0001).

The reciprocal increase in the proportion of different memory and effector subsets varied across the CD4+ and CD8+ T cells subsets. In CD4+ T cells, the proportion of central memory T cells increased with age (FIG. 1B, p<0.0001) but in the older adult groups, there was a significant decline in the proportion of effector memory (CD45RA−CCR7−) and effector (CD45RA+CCR7−) CD4+ T cells in the >80 yo age group relative to the 60-70 yo age group (FIG. 1B). In contrast, CD8+ T cell subsets (FIG. 1C) showed an age-related increase in the proportion of central memory (p<0.0001), effector memory (p<0.0001) and effector (p=0.001) T cells. These results show that contraction of the naïve T cell pool with aging is associated with CD4+ T cell expansion mainly in the central memory subset. In contrast, CD8+ memory T cells are more differentiated to effector memory and effector T cells, which is consistent with aging having its greatest impact on the CD8+ T cell subset.

### Example 2

### Effect of Age on Effector T Cells Expressing GzmB and Perforin

Previous studies have shown that perforin (Perf) and GzmB are key cytolytic effector molecules, which are stored in the granules of cytolytic T cells [18, 19]. Upon T cell receptor binding to the peptide-MHC I complex, granules containing granzymes and Perf migrate to the cell surface of and are released from the CTL. Perf facilitates the entry of GzmB into virus-infected host cells to cause apoptotic cell death, and thus is necessary for effective cytolytic activity.

The next experiments evaluated the effect of age on the cytolytic effector function of different T-cell subsets. The intracellular expression of effector molecules, GzmB and Perf, in different CD4+ or CD8+ T-cell subsets (FIG. 1A) was analyzed to estimate their cytolytic potential in response to live influenza virus.

In CD4+ T cells, GzmB was expressed only in the effector (CD45RA+CCR7−) subset (FIG. 1D), while in CD8+ T cells, GzmB was expressed in a subset of all memory and effector cells (FIG. 1E), as has been previously shown [20]. The proportion of GzmB+ cells increased with age in these virus-stimulated CD4+ and CD8+ T cells subsets (FIGS. 1D and 1E) but the proportion was greater in CD8+ T cells and there was a plateau with no further increase from the 60-70 yo to the >80 yo age group (FIGS. 1D and 1E). However, previous results showed that a large proportion of CD45RA+CD8+ T cells from older adults express GzmB at baseline and these GzmB+CD8+ T cells respond poorly to virus stimulation [8], consistent with a terminally differentiated phenotype [1]. To further distinguish the phenotype of cytolytic T cells responding to influenza virus, these T cell subsets for the co-expression of GzmB and Perf in influenza-stimulated PBMC were evaluated. A response to vaccination was detected but the increase in the proportions of GzmB+Perf+ T cells in CD4+ and CD8+ subsets was not statistically significant due to the high variability in the proportions of GzmB+Perf+ T cells in pre-vaccination samples (FIG. 5). At 4-weeks post-vaccination, the proportion of effector CD4+ T cells that co-expressed GzmB and Perf was similar across the three age groups and to the proportion of GzmB+ effector CD4+ T cells in young adults (FIG. 1D).

In contrast, the CD8+ T cell subset showed a statistically significant reduction in the proportion of effector T cells that were GzmB+Perf+ compared to GzmB+ alone (FIG. 1E, p<0.0001). While young and older adults had similar proportions of GzmB+Perf+ effector CD8+ T cells at 4-weeks post-vaccination, there was a significant age-related decline in the proportion of GzmB+Perf+ effector T cells by 10-weeks post-vaccination (FIG. 1F), which was more marked in the CD8+ T cells compared to CD4+ T cells. These results show an age-related shift in the relative proportion of GzmB+Perf+ effector T cells responding to influenza challenge within the CD4+ and CD8+ subsets such that CD4+ T cells are the main source of effector T cells by 10-weeks post-vaccination in older adults. Since the influenza season usually occurs after 10-weeks post-vaccination, these data show that this decline in the effector T cell response to influenza challenge is clinically important with respect to serious outcomes of influenza infection [21].

### Example 3

### Degranulation Markers of Cytolytic Effector Function in Response to Virus Stimulation

Previous experiments showed that older adults (mainly those with congestive heart failure) who showed an increased proportion of GzmB+CD8+ T cells expressing the degranulation marker, CD107a, at baseline, mounted a poor response to influenza virus stimulation [8, 22]. In contrast, healthy older compared to young adults showed an increased proportion of GzmB+CD107a+CD8+ T cells in the response to influenza virus. Thus, it was of interest to determine whether the relative changes in GzmB+Perf+ T cells within the CD4+ and CD8+ subsets of influenza-stimulated PBMC could be detected using CD107a.

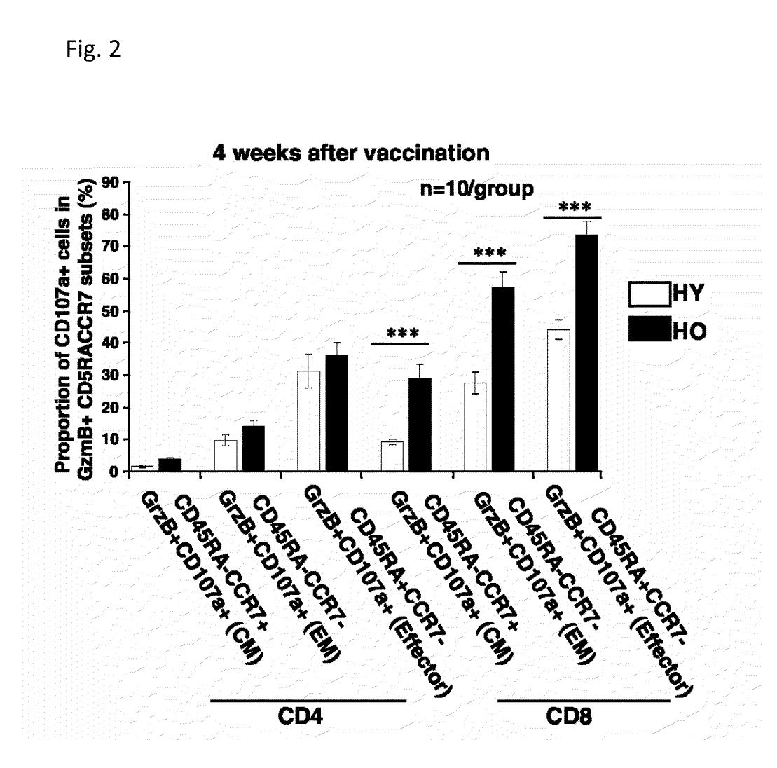

As in a previous study, healthy young and older adults showed similar proportions of CD8+ T cells (and CD4+ T cells in this study) that were CD107a+ after 12 hours of virus stimulation. Thus, it was of interest to measure degranulating activity in response to live influenza virus (12-hour stimulation) within each of the memory and effector T-cell subsets using cell surface expression of CD107a (FIG. 2). Young and older adult PBMC showed similar proportions of degranulating cells (CD107a+) within the memory and effector GzmB+CD4+ T− cell subsets (FIG. 2). However, older compared to young adults showed a significant increase in the proportion of GzmB+CD8+ T cells expressing CD107a; the increase was >20% in the central memory and >30% in effector memory and effector subsets (p<0.001 for all comparisons) (FIG. 2). These results show that a larger proportion of CD8+ T cells were activated to mount a cytolytic response to influenza virus challenge. A functional assay of cytolytic activity confirmed these results.

### Example 4

### Cytolytic Activity in Virus-Specific CD4+ and CD8+Effector T-Cell Subsets

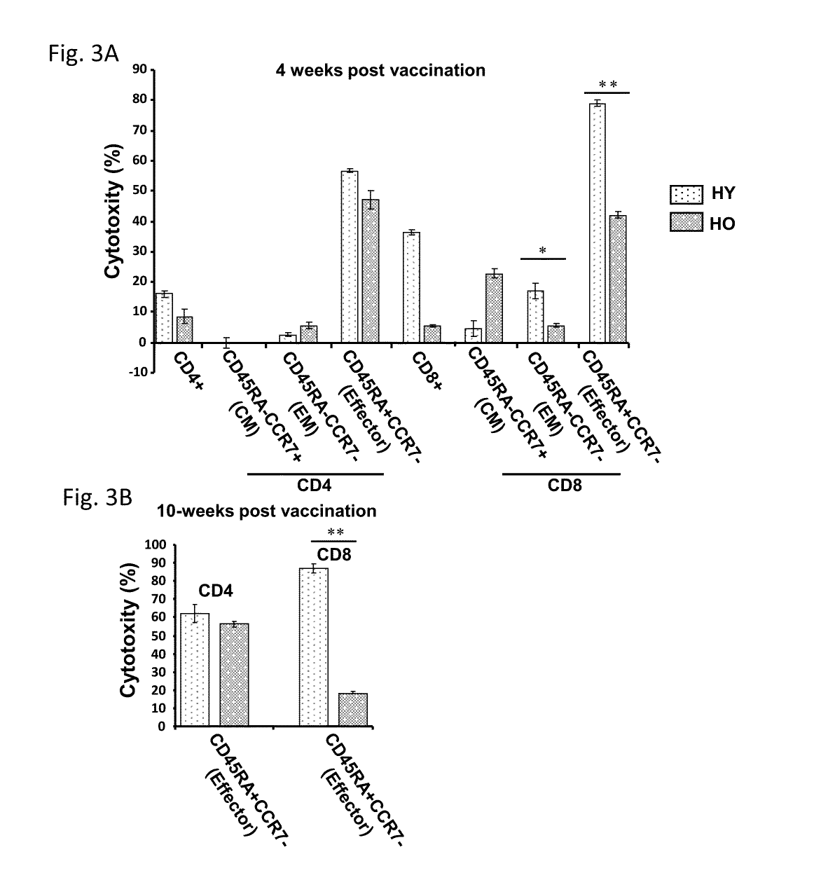

To further show the cytolytic function of different T-cell subsets responding to influenza virus, cytotoxicity assays were performed. CD4+ or CD8+ T cells were stimulated with H3N2, sorted into the different memory and effector T cells subsets, and then used as effector cells (E) in cytotoxicity assays with PHA-stimulated, autologous PBMCs infected with influenza virus as target cells (T) (FIG. 3). Results are reported as % specific lysis at an E:T ratio=10:1. There was no cytolytic activity detected in unstimulated PBMC. In virus-stimulated CD4+ T cells from young and older (60-75 years old) adults, similar levels of cytolytic activity in the effector (CD45RA+CCR7−) and effector memory (CD45RA−CCR7−) subsets at 4 weeks (FIG. 3A) and 10-weeks (FIG. 3B) post-vaccination were found. In contrast, young compared to older adults exhibited significantly higher cytolytic activity in CD8+ T cells within the effector memory (p<0.01) and effector (p<0.005) subsets at 4-weeks post-vaccination (FIG. 3A).

By 10 weeks post-vaccination, there was a significant further decline in influenza virus specific cytolytic activity of effector CD8+ T cells in older compared to young adults (FIG. 3B, p<0.005). A corresponding diminished proliferative response in older compared to young adults after five days of stimulation with the A/H3N2 strain was demonstrated in the CD8+ effector T cell subset (FIG. 6).

Taken together, these results show that a split (killed) virus vaccine stimulates a more sustained memory T cell response in the CD4+ relative to the CD8+ subset. While similar proportions of GzmB+Perf+ effector (CD45RA+CCR7−) T cells can be stimulated in both CD4+ and CD8+ T cell subsets in young and older adults at 4-weeks post vaccination, there is a decline in this effector CD8+ T cell subset by 10-weeks post vaccination in older adults. In addition, significantly lower levels of cytolytic activity in effector CD8+ T cells in older compared to young adults at 4-weeks post-vaccination, and a further reduction in older adults by 10 weeks post-vaccination are evidence of functional defects in the CD8+ T cell response. In contrast, cytolytic activity in CD4+ effector T cells was similar in young and older adults and sustained from 4-weeks to 10-weeks post-vaccination in effector CD4+ T cells; although the frequency of this T cell subset declined by 10-weeks post-vaccination in older adults but was not as marked as in the CD8+ T cell subset. Overall, a more sustained memory CD4+ T cell response was elicited by influenza vaccination in older adults while the response to vaccination was sustained in both CD4+ and CD8+ subsets in young adults.

### Example 5

### Phenotype of Virus-Specific Cytotoxic T Cells in Vaccinated Young and Older Adults

In this study, T-cells subsets and their effector function based on T-cell surface markers CD45RA and CCR7 as well as effector molecules, GzmB and Perf, and related cytolytic activity were analyzed. It was found that effector T cells that become GzmB+Perf+ in response to influenza challenge are the T cells with high cytolytic activity against influenza-infected targets. The phenotype of the GzmB+Perf+ T cells responding to influenza stimulation was further characterized. Since the expression of IL-7 receptor alpha (IL-7Ralpha) can distinguish functional subsets of CD8+ T cells specific for different respiratory viruses in humans [23], an antibody to CD127 (IL-7Ralpha) was used in this analysis. In the study, most of the effector T cells (GzmB+Perf+) expressed CD127 and could be further separated into two groups based on expression of the surface marker, CD25 (IL-2 receptor) (FIG. 4A). The CD25+ effector T-cell subset that was identified has the phenotype, CD45RA+CCR7− CD25+CD127+, which is consistent with high proliferative capacity in response to A/H3N2 compared to pH1N1 in the CD25+ subset, and to both strains in the CD25− subset (FIG. 4D). Comparable levels of cytolytic activity on a per cell basis were generated in the CD25+ subset in response to H3N2 and pH1N1 strains (FIG. 4E), whereas the CD25− subset had ˜50% of the cytolytic activity in the CD25+ subset.

### Example 6

### Effector T Cell Response to A/H3N2 vs. pH1N1 after Seasonal Influenza Vaccination

In this study, the proliferative and cytolytic T cell response to A/H3N2 and pH1N1 within the effector T-cell subset (CD45RA+CCR7−) was compared. Since A/H3N2, A/H1N1, and pH1N1 strains share some T cell epitopes within the internal proteins derived from the virus [20], seasonal vaccine could stimulate a T cell response to A/H3N2 and pH1N1 strains. These experiments specifically focused on the phenotype of the effector T-cell subset after 20 hours of virus stimulation, and the proliferative and CTL response after in vitro stimulation (5-6 days) with live influenza virus. No changes in the T cell phenotypes were observed from 20 hours to 5-6 days of in vitro stimulation. All CD45RA+CCR7− GzmB+Perf+ T cells were CD127+ but there was a significantly lower proportion of effector T cells that were CD25+ in pH1N1 vs. A/H3N2-stimulated PBMC (p<0.001) (FIGS. 4A and 4B, p<0.001), and a corresponding higher proportion of CD25− effector T cells in both CD4+ and CD8+ subsets (FIG. 4C). The lower proliferative response stimulated by the pH1N1 compared to the A/H3N2 strains is demonstrated in the CD25+ subsets of CD4+ and CD8+ effector T cells from young adults (FIG. 4D), and is associated with lower cytolytic activity within the effector (CD45RA+CCR7−) subsets of CD4+(p<0.01) and CD8+(p<0.005) T cells (FIG. 4E). The CD25+CD127+ effector subsets of CD4+ or CD8+ T cells from young adults exhibited similar levels of cytolytic activity against A/H3N2 and pH1N1-infected targets following stimulation with live virus (FIG. 4E). The proportion of CD25+CD127+ effector T cells responding to A/H3N2 and pH1N1 strains significantly declined with advancing age group in both the CD4+ (p<0.001) and CD8+(p<0.001) T-cell subsets (FIG. 4F). Compared to the influenza A/H3N2 strain, there was a very limited CD25+ effector CD8+ T cell response to pH1N1 stimulation in both older age groups with no statistical difference observed between the two subtypes (less than 3% of total effector GzmB+Perf+ T cells) (FIG. 4F).

These results show that the main phenotype of CD4+ and CD8+ T cells responding to influenza virus are within the CD45RA+CCR7−CD25+CD127+ subset. In other words, these are the cells that express IL-2R (CD25) and thus have recently undergone proliferation and have the highest level of cytolytic activity. Once these T cells become CD25−, their cytolytic activity declines to approximately 50% of the CD25+ effector T cells showing that they are becoming exhausted. The expression of IL-7R (CD127) is consistent with the phenotype of influenza-specific CD8+ T cells in PBMC [24] and presumably the phenotype of T cells responding to dendritic cells or macrophages presenting influenza-derived peptides in PBMC cultures. Activation of these CD8+ T cells in the lungs has been characterized by a reduction in the expression of IL-7R [24]. These results show that the phenotype of cytolytic effectors responding in vitro to influenza is CD45RA+CCR7−CD25+CD127+ in both CD4+ and CD8+ T cell subsets.

### Example 7

### Baseline GrzB Activity in Older Adults

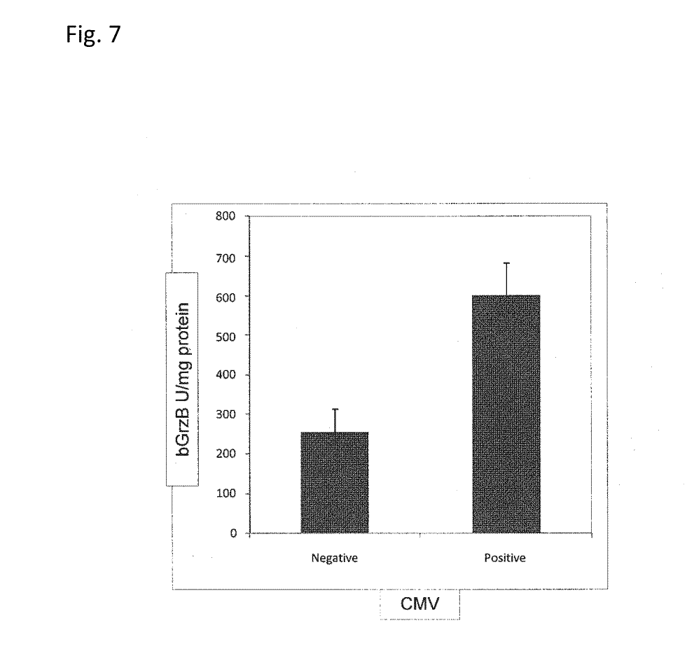

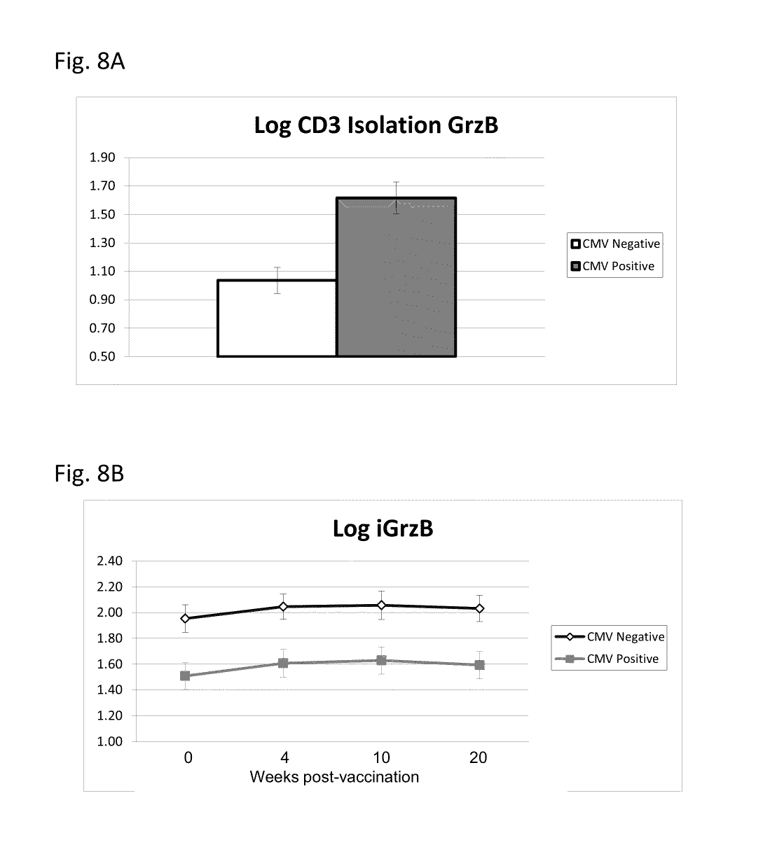

A population of older adults, both healthy, and previously diagnosed with diabetes were assessed for their human cytomegalovirus (CMV) serum antibody status, and for GrzB activity in unstimulated PBMC lysates. CMV status was determined by serology using conventional assays. 21 subjects were found to be CMV positive (CMV+), 7 subjects were CMV negative (CMV−). CMV+ subjects demonstrated significantly higher GrzB activity in the unstimulated PBMC lysates (baseline, or bGrzB activity). See. FIG. 7. The presence of diabetes did not affect bGzmB activity in this analysis. In a regression analysis including bGrzB as the dependent variable, and CMV serologic status (CMV+vs. CMV−) and diabetes (presence of diabetes vs. no diabetes) as the independent variables, only CMV serologic status was statistically significant. FIG. 8.A. shows the results in the next year of the study, which reproduced the results shown in FIG. 7. In this regard, GrzB activity in unstimulated CD3+ T cells (bGrzB) is significantly higher in CMV+vs. CMV− (p=0.001). FIG. 8.B. shows iGrzB activity (fold increase in total GrzB activity over bGrzB activity) in response to influenza challenge in pre- and 4-, 10- and 20-week post-vaccinatio PBMC. Influenza challenge induces significantly higher levels of iGrzB activity in CMV− vs CMV+ older adults (p=0.01). The presence of diabetes was not significant in either analysis.

### Example 8

### Background GrzB Activity and Incidence of Influenza in CMV+ and CMV− Subjects

Chronic CMV infection leads to T cell exhaustion and accumulation of GrzB+ T cells that cannot respond to viral infection. These T cells deposit GrzB in immune tissue, resulting in inflammation and destruction of tissues of the immune system, which leads to suppression of an effective immune response to an infection, for example an influenza infection. Age cohorts of subjects (10 to 80+ years old) are recruited, and serum samples obtained (before and after vaccination and/or exposure to influenza) to determine CMV status (seropositive or seronegative) and GrzB activity in peripheral blood leukocytes and plasma. Subjects are followed in the clinic to determine the onset and/or incidence of influenza, and/or development of influenza symptoms (e.g. fever). The relationship between CMV status, GrzB activity, and vaccine responsiveness and efficacy (scored by onset and/or incidence of influenza, and/or development of influenza symptoms) are described in Examples 9 and 10.

### Example 9

PBMC collected pre-vaccination and at 4-, 10-, and 20-weeks post-vaccination are stimulated with live influenza virus for 20 hours and lysates prepared for assay of total GrzB (tGrzB) activity in the lysate. To determine bGrzB activity, CD3+ T cells are purified by magnetic bead selection from unstimulated PBMC collected pre-vaccination, and lysates prepared for assay of baseline levels of GrzB activity (this level of bGrzB activity remains unchanged over time when health status remains stable). A regression analysis is used to determine the amount of variance in the change in iGrzB levels in influenza-stimulated PBMC from pre-vaccination to 4-weeks post-vaccination (R²=0.421 in this case) that is predicted by bGrzB levels (see, for example, FIG. 8). The bGrB activity at the time of vaccination is predictive of up to 42.1% (p=0.002) of the variation in the iGrzB response to vaccination (the change in iGrzB activity from pre-vaccination to post vaccination). In some embodiments of the invention age is a significant predictor of the ability to exhibit a cell-mediated immune response—this is supportive of the significant association of iGrzB activity with the efficacy of the cell mediated immune response to administration of an immunostimulatory composition.

### Example 10

Regression analysis showed that CMV seropositive (CMV+) vs. seronegative (CMV−) status and study group (healthy young vs. healthy old vs. old COPD (chronic obstructive pulmonary disease) vs. old CHF (congestive heart failure)) predicted bGrzB activity (R=0.527) with health status (increasing age and chronic disease, B=−0.224, p<0.0001) and CMV+ status (B=0.713, p<0.0001) being associated with a significant increase in bGrzB activity. Further, iGrzB calculated as the fold increase in GrzB activity over baseline (bGrzB) in response to influenza challenge following 5-day culture with vaccine, similarly predicted the response to the SVV (Split-virus vaccine). In the model, which included study group, CMV status and effect of treatment (SVV vs. no SVV), there was a significant correlation between the iGrzB response to influenza challenge (R=0.612); health status (increasing age and chronic disease, B=−0.282, p<0.0001), effect of SVV (B=1.032, p<0.0001) and CMV+ status (B=−0.775, p<0.0001). In summary, both CMV and age/health status predict an increase in the potentially damaging effects of extracellular GrzB (measured by bGrzB), in this case, affecting the overall response to influenza challenge: SVV has a positive effect while CMV+ status and older age/chronic disease negatively impact on the response to influenza challenge.

### REFERENCES

1. Pawelec G, Akbar A, Caruso C, Solana R, Grubeck-Loebenstein B, Wikby A. Human immunosenescence: is it infectious? Immunol Rev 2005 June; 205:257-68.
2. Thompson W W, Shay D K, Weintraub E, Brammer L, Cox N, Anderson U, et al. Mortality associated with influenza and respiratory syncytial virus in the United States. Jama 2003 Jan. 8; 289(2):179-86.
3. Thompson W W, Shay D K, Weintraub E, Brammer L, Bridges C B, Cox N J, et al. Influenza-associated hospitalizations in the United States. Jama 2004 Sep. 15; 292(11):1333-40.
4. Update: influenza activity—United States, Aug. 30, 2009-Jan. 9, 2010. MMWR Morb Mortal Wkly Rep; 59:38-43.
5. Outbreaks of 2009 Pandemic Influenza A (H1N1) Among Long-Term—Care Facility Residents—Three States, 2009. MMWR Morb Mortall Wkly Rep; 59:74-7.
6. Nichol K L, Margolis K L, Wuorenma J, Von Sternberg T. The efficacy and cost effectiveness of vaccination against influenza among elderly persons living in the community. N Engl J Med 1994 Sep. 22; 331(12):778-84.
7. McElhaney J E, Xie D, Hager W D, Barry M B, Wang Y, Kleppinger A, et al. T cell responses are better correlates of vaccine protection in the elderly J Immunol 2006 May 15; 176(10):6333-9.
8. McElhaney J E, Ewen C, Zhou X, Kane K P, Xie D, Hager W D, et al. Granzyme B: Correlates with protection and enhanced CTL response to influenza vaccination in older adults. Vaccine 2009 Apr. 21; 27(18):2418-25.
9. McMichael A J, Gotch F M, Noble G R, Beare P A. Cytotoxic T-cell immunity to influenza. N Engl J Med 1983 Jul. 7; 309(1):13-7.
10. Johnson B J, Costelloe E O, Fitzpatrick D R, Haanen J B, Schumacher T N, Brown L E, et al. Single-cell perforin and granzyme expression reveals the anatomical localization of effector CD8+ T cells in influenza virus-infected mice. Proc Natl Acad Sci USA 2003 Mar. 4; 100(5):2657-62.
11. Lord S J, Rajotte R V, Korbutt G S, Bleackley R C. Granzyme B: a natural born killer. Immunol Rev 2003 June; 193:31-8.
12. Lawrence C W, Ream R M, Braciale T J. Frequency, specificity, and sites of expansion of CD8+ T cells during primary pulmonary influenza virus infection J Immunol 2005 May 1; 174(9):5332-40.
13. Pfister G, Weiskopf D, Lazuardi L, Kovaiou R D, Cioca D P, Keller M, et al. Naive T cells in the elderly: are they still there? Ann N Y Acad Sci 2006 May; 1067:152-7.
14. McElhaney J E, Pinkoski M J, Upshaw C M, Bleackley R C. The cell-mediated cytotoxic response to influenza vaccination using an assay for granzyme B activity J Immunol Methods 1996 Mar. 28; 190(1):11-20.
15. Powers D C, McElhaney J E, Florendo O A, Jr., Manning M C, Upshaw C M, Bentley D W, et al. Humoral and cellular immune responses following vaccination with purified recombinant hemagglutinin from influenza A (H3N2) virus. J Infect Dis 1997 February; 175(2):342-51.
16. Sallusto F, Lenig D, Forster R, Lipp M, Lanzavecchia A. Two subsets of memory T lymphocytes with distinct homing potentials and effector functions. Nature 1999 Oct. 14; 401(6754):708-12.
17. Olsson J, Wikby A, Johansson B, Lofgren S, Nilsson B O, Ferguson F G. Agerelated change in peripheral blood T-lymphocyte subpopulations and cytomegalovirus infection in the very old: the Swedish longitudinal OCTO immune study. Mech Ageing Dev 2000 Dec. 20; 121(1-3):187-201.
18. Russell J H, Ley T J. Lymphocyte-mediated cytotoxicity. Annu Rev Immunol 2002; 20:323-70.
19. Barry M, Bleackley R C. Cytotoxic T lymphocytes: all roads lead to death. Nat Rev Immunol 2002 June; 2(6):401-9.
20. Touvrey C, Derre L, Devevre E, Corthesy P, Romero P, Rufer N, et al. Dominant human CD8 T cell clonotypes persist simultaneously as memory and effector cells in memory phase. J Immunol 2009 Jun. 1; 182(11):6718-26.
21. Shahid Z, Kleppinger A, Gentleman B, Falsey A R, McElhaney J E. Clinical and immunologic predictors of influenza illness among vaccinated older adults. Vaccine 2010 Aug. 31; 28(38):6145-51.
22. Betts M R, Brenchley J M, Price D A, De Rosa S C, Douek D C, Roederer M, et al. Sensitive and viable identification of antigen-specific CD8+ T cells by a flow cytometric assay for degranulation. Journal of Immunological Methods 2003 Oct. 1; 281(1-2):65-78.
23. van Leeuwen E M, de Bree G J, Remmerswaal E B, Yong S L, Tesselaar K, ten Berge I J, et al. IL-7 receptor alpha chain expression distinguishes functional subsets of virus-specific human CD8+ T cells. Blood 2005 Sep. 15; 106(6):2091-8.
24. de Bree G J, van Leeuwen E M, Out T A, Jansen H M, Jonkers R E, van Lier R A. Selective accumulation of differentiated CD8+ T cells specific for respiratory viruses in the human lung. J Exp Med 2005 Nov. 21; 202(10):1433-42.
25. Effros R B. Role of T lymphocyte replicative senescence in vaccine efficacy. Vaccine 2007 Jan. 8; 25(4):599-604.
26. Jain S, Kamimoto L, Bramley A M, Schmitz A M, Benoit S R, Louie J, Sugerman D E, Druckenmiller J K, Ritger K A, Chugh R, Jasuja S, Deutscher M, Chen S, Walker J D, Duchin J S, Lett S, Soliva S, Wells E V, Swerdlow D, Uyeki T M, Fiore A E, Olsen S J, Fry A M, Bridges C B, Finelli L; 2009 Pandemic Influenza A (H1N1) Virus Hospitalizations Investigation Team. Hospitalized patients with 2009 H1N1 influenza in the United States, April-June 2009. N Engl J Med. 2009 Nov. 12; 361(20):1935-44.
27. Goronzy J J, Fulbright J W, Crowson C S, Poland G A, O'Fallon W M, Weyand C M. Value of immunological markers in predicting responsiveness to influenza vaccination in elderly individuals. J Virol 2001 December; 75(24):12182-7.
28. Saurwein-Teissl M, Lung T L, Marx F, Gschosser C, Asch E, Blasko I, et al. Lack of antibody production following immunization in old age: association with CD8(+)CD28(−) T cell clonal expansions and an imbalance in the production of Th1 and Th2 cytokines. J Immunol 2002 Jun. 1; 168(11):5893-9.
29. Effros R B, Dagarag M, Spaulding C, Man J. The role of CD8+ T-cell replicative senescence in human aging. Immunol Rev 2005 June; 205:147-57.
30. Casazza J P, Betts M R, Price D A, Precopio M L, Ruff L E, Brenchley J M, et al. Acquisition of direct antiviral effector functions by CMV-specific CD4+ T lymphocytes with cellular maturation. J Exp Med 2006 Dec. 25; 203(13):2865-77.
31. Sylwester A W, Mitchell B L, Edgar J B, Taormina C, Pelte C, Ruchti F, et al. Broadly targeted human cytomegalovirus-specific CD4+ and CD8+ T cells dominate the memory compartments of exposed subjects. J Exp Med 2005 Sep. 5; 202(5):673-85.
32. de Bree G J, Daniels H, Schilfgaarde M, Jansen H M, Out T A, van Lier R A, et al. Characterization of CD4+ memory T cell responses directed against common respiratory pathogens in peripheral blood and lung. J Infect Dis 2007 Jun. 1; 195(11):1718-25.
33. de Bree G J, Heidema J, van Leeuwen E M, van Bleek G M, Jonkers R E, Jansen H M, et al. Respiratory syncytial virus-specific CD8+ memory T cell responses in elderly persons. J Infect Dis 2005 May 15; 191(10):1710-8.
34. Kang I, Hong M S, Nolasco H, Park S H, Dan J M, Choi J Y, et al. Age-associated change in the frequency of memory CD4+ T cells impairs long term CD4+ T cell responses to influenza vaccine. J Immunol 2004 Jul. 1; 173(1):673-81.
35. Gardner E M, Gonzalez E W, Nogusa S, Murasko D M. Age-related changes in the immune response to influenza vaccination in a racially diverse, healthy elderly population. Vaccine 2006 Mar. 6; 24(10):1609-14.
36. Pebody R G, McLean E, Zhao H, Cleary P, Bracebridge S, Foster K, et al. Pandemic Influenza A (H1N1) 2009 and mortality in the United Kingdom: risk factors for death, April 2009 to March 2010. Euro Surveill 2010 May 20; 15(20).
37. Hadler J L, Konty K, McVeigh K H, Fine A, Eisenhower D, Kerker B, et al. Case fatality rates based on population estimates of influenza-like illness due to novel H1N1 influenza: New York City, May-June 2009. PloS one 2010; 5(7):e11677.
38. Skowronski D M, Hottes T S, Janjua N Z, Purych D, Sabaiduc S, Chan T, et al. Prevalence of seroprotection against the pandemic (H1N1) virus after the 2009 pandemic. Cmaj 2010 Nov. 23; 182(17):1851-6.
39. Hammarlund E, Lewis M W, Hansen S G, Strelow L I, Nelson J A, Sexton G J, et al. Duration of antiviral immunity after smallpox vaccination. Nature medicine 2003 September; 9(9):1131-7.
40. Amara R R, Nigam P, Sharma S, Liu J, Bostik V. Long-lived poxvirus immunity, robust CD4 help, and better persistence of CD4 than CD8 T cells. Journal of virology 2004 April; 78(8):3811-6.
41. Combadiere B, Boissonnas A, Carcelain G, Lefranc E, Samri A, Bricaire F, et al. Distinct time effects of vaccination on long-term proliferative and IFN-gamma-producing T cell memory to smallpox in humans. The Journal of experimental medicine 2004 Jun. 7; 199(11):1585-93.
42. Shahid Z, Kleppinger A, Gentleman B, Falsey A R, McElhaney J E. Clinical and immunologic predictors of influenza illness among vaccinated older adults. Vaccine 2010 Aug. 31; 28(38):6145-51.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1.-28. (canceled)

29. A method for determining an immune response of a subject to an immunostimulatory composition, the method comprising:
(a) determining a first level of granzyme B (bGrzB) activity in a first sample from the subject,
(b) administering the immunostimulatory composition to the subject,
(c) determining a second level of granzyme B (tGrzB) activity in a second sample from the subject, and
(d) calculating an induced level of granzyme B (iGrzB) activity, wherein iGrzB activity=fold increase in tGrzB activity over bGrzB activity,
wherein, a high level of bGrzB activity, a low level of iGrzB activity or a low level of tGrzB activity indicates that the subject is at risk of developing a poor immune response to the immunostimulatory composition, and
wherein a low level of bGrzB activity or a high level of iGrzB activity indicates that the subject is at risk of developing a strong immune response to the immunostimulatory composition.

30. The method according to claim 29, wherein the low level of tGrzB activity is less than about 990 U/mg.

31. The method according to claim 29, wherein the high level of bGrzB activity is greater than about 300 U/mg, preferable greater than about 600 U/mg, and the low level of bGrzB activity is less than about 300 U/mg.

32. The method according to claim 29, wherein the high level of tGrzB activity is greater than about 990 U/mg.

33. The method according to claim 29, wherein the high level of iGrzB activity has a base 10 log iGrzB value that is greater than 1.9, optionally greater than 2, and wherein the low level of iGrzB activity has a base 10 log iGrzB value that is less than 1.8, optionally, less than 1.7.

34. (canceled)

35. The method according to claim 29, wherein the immune response is a cell mediated immune response, preferably a CD8 T cell mediated immune response.

36. (canceled)

37. A method of determining an immune response of a subject to an immunostimulatory composition, the method comprising:
(a) immunizing the subject with the immunostimulatory composition,
(b) determining a first level of granzyme B (bGrzB) activity in a test sample from the subject,
(c) stimulating the test sample with the immunostimulatory composition,
(d) determining a second level of granzyme B (tGrzB) activity from the test sample stimulated with the immunostimulatory composition,
(e) calculating an induced level of granzyme B (iGrzB) activity, wherein iGrzB activity is equal to fold increase in tGrzB activity over bGrzB activity, and
(f) comparing the iGrzB activity in the test sample to an iGrzB activity of a control sample,
wherein a decrease in the level of bGrzB activity and/or an increase in the level of iGrzB activity in the test sample compared to the control sample indicates development of a strong immune response in the subject to the immunostimulatory composition, and
wherein a similar or an increase in the level of bGrzB activity and/or a similar or a decrease in the level iGrzB activity in the test sample compared to the control sample indicates development of a poor immune response in the subject to the immunostimulatory composition.

38. The method according to claim 37, wherein the level of bGrzB activity in the test sample compared to the control sample that is indicative of development of a strong immune response in the subject to the immunostimulatory composition is decreased by at least 1.5 fold.

39. The method according to claim 37, wherein the level of iGrzB activity in the test sample compared to the control sample that is indicative of development of a strong immune response in the subject to the immunostimulatory composition is increased by at least 1.3 fold.

40. The method according to claim 37, wherein the control sample represents subjects who are chronic disease positive.

41. The method according to claim 40, wherein the chronic disease is congestive heart failure, COPD, CMV infection, HIV infection, Epstein Barr infection, or Herpes zoster infection.

42. The method according to claim 37, wherein the control sample represents subjects who are about 60 years of age, or older.

43.-47. (canceled)

48. A method of treating a subject in need thereof, the method comprising
(a) determining a level of granzyme B activity in a test sample from the subject, and
(b) if the level of granzyme B activity in the test sample is greater than a level of granzyme B activity in a chronic disease negative population, then
(i) treating the subject with an enhanced immunostimulatory composition, and/or
(ii) treating the subject with an immunostimulatory composition according to an altered immunization schedule, and/or
(iii) treating the subject with a therapeutic agent.

49. The method according to claim 48, wherein the altered immunization schedule is no more than four weeks before an annually-recurring time period characterized by the prevalence of outbreaks of the pathogen to which the immunostimulatory composition is directed.

50. The method according to claim 48, wherein the chronic disease is a chronic CMV infection.

51. The method according to claim 48, wherein the enhanced immunostimulatory composition comprises the immunostimulatory composition and an adjuvant, or comprises a high dose formulation of the immunostimulatory composition.

52. (canceled)

53. The method according to claim 48, wherein the therapeutic agent is an antiviral agent, such as Oseltamivir or Zanamivir.

54. The method according to claim 51, wherein the adjuvant is glucopyranosyl lipid adjuvant-stable emulsion (GLA-SE), resiquimod, poly I:C nucleic acid molecule, CpG nucleic acid molecule, polyIC nucleic acid molecule, polyICLC nucleic acid molecule, polyIC/R nucleic acid molecule, aluminum hydroxide, alum, aluminum trihydrate, virosome, squalene, oils, MF59, QS21, saponin, virus-like particles, monophosphoryl-lipidA/trehalose dicorynomycolate, polyoxypropylene or polyoxyethylene.

55. The method according to claim 48, wherein the immunostimulatory composition is an influenza vaccine, preferably, a seasonal influenza vaccine or a pandemic influenza vaccine, wherein the influenza vaccine is a commercial formulation of influenza vaccine (preferably Fluviral, Vaxigrip, or Fluzone), optionally a split virus vaccine.

56.-58. (canceled)

59. The method according to claim 48, wherein the subject is a human.

60.-63. (canceled)
